(19) United States
(12) Patent Application Publication
(10) Pub. No.: US 2008/0274096 A1
(43) Pub. Date: Nov. 6, 2008

(54) Fusion Proteins Having a Modulated Half-Life in Plasma

(75) Inventors: Christin Andersson, Sodertalje (SE); Per-Ola Freskgard, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/089,127

(22) PCT Filed: Oct. 2, 2006
(86) PCT No.: PCT/SE2006/001113

§ 371 Date: May 30, 2008

(30) Foreign Application Priority Data
Oct. 3, 2005 (SE) 0502175-3

(51) Int. Cl. A61K 38/48; C12N 9/50; A61P 25/28; A61P 9/00; C07K 1/14

(52) U.S. Cl. 424/94.63; 435/219; 435/269; 435/272

(57) ABSTRACT

The present invention relates to fusion proteins and their use in enzymatic treatment of Alzheimer's disease patients. Said fusion protein comprises a component that cleaves the amyloid beta (Ab) peptide e.g. neprilysin, insulin degrading enzyme (IDE), another component that modulates the half-life in plasma e.g. the Fc portion of IgG or PEG; and a third component that connects the first two components.

The present invention relates fusion proteins and their use in enzymatic treatment of Alzheimer's disease patients. Said fusion protein comprises a component that cleaves the amyloid beta (Aβ) peptide, another component that modulates the half-life in plasma; and a third component that connects the first two components.

### BACKGROUND OF THE INVENTION

The present invention relates to methods of preventing amyloid plaque formation and/or growth by reacting amyloid peptides with an enzyme that specifically recognizes amyloid peptides, and inactivates them through degradation or modification. The present invention in further relates to a method of treating Alzheimer's disease by administering an optimized amyloid peptide-degrading enzyme with improved catalytic activity and/or selectivity. The present invention also relates to the field of medical therapy, in particular to the field of neurodegenerative disease and provides methods of eliciting clearance mechanisms for brain amyloid in patients suffering from neurodegenerative diseases, in particular Alzheimer's disease. Furthermore, this invention relates to the use of proteins and peptides effective in eliciting such mechanisms.

The present invention describes how an Aβ-peptide degrading molecule can become a therapeutic relevant agent by attaching a molecule that modulates the stability and half-life in blood plasma. The Aβ-peptide degrading molecules describe in this invention overall posseses to short plasma half-life to be useful as an effective therapeutic agent. However, by combining these degrading molecules with the described and exemplified modulator molecules in this invention, functional agents is produced that can be used effectively in treating Alzheimer's disease by administering these optimized amyloid peptide-degrading enzyme fusion protein.

Neurodegenerative diseases, in particular Alzheimer's disease (AD), have a strong debilitating impact on a patient's life. Furthermore, these diseases constitute an enormous health, social and economic burden. AD is the most common age-related neurodegenerative condition affecting about 10% of the population over 65 years of age and up to 45% over age 85 (Vickers et al., Progress in Neurobiology 2000, 60:139-165). Presently, this amounts to an estimated 12 million cases in the US, Europe, and Japan. This situation will inevitably, worsen with the demographic increase in the number of old people in developed countries. The neuropathological hallmarks that occur in the brain of individuals suffering from AD are senile plaques and profound cytoskeletal changes coinciding with the appearance of abnormal filamentous structures and the formation of neurofibrillary tangles. Both familial and sporadic cases share the deposition in brain of extracellular, fibrillary β-amyloid as a common pathological hallmark that is believed to be associated with impairment of neuronal functions and neuronal loss (Younkin S. G., Ann. Neurol. 37, 287-288, 1995; Selkoe, D. J., Nature 399, A23-A31, 1999; Borchelt D. R. et al., Neuron 17, 1005-1013, 1996). β-amyloid deposits are composed of several species of amyloid-β peptides (Aβ); especially Aβ₄₂ is deposited progressively in amyloid plaques. AD is a progressive disease that is associated with early deficits in memory formation and ultimately leads to the complete erosion of higher cognitive function. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10:184-192).

Genetic evidence suggests that increased amounts of Aβ₄₂ are produced in many, if not all, genetic conditions that cause familial AD (Borchelt D. R. et al., Neuron 17, 1005-1013, 1996; Duff K. et al., Nature 383, 710-713, 1996; Scheuner D. et al., Nat. Med. 2, 864-870, 1996; Citron M. et al., Neurobiol. Dis. 5, 107-116, 1998), pointing to the possibility that amyloid formation may be caused either by increased generation of Aβ₄₂, or decreased degradation, or both (Glabe, C., Nat. Med. 6, 133-134, 2000). Although these are rare examples of early-onset AD, which have been attributed to genetic defects in the genes for APP, presenilin-1, and presenilin-2, the prevalent form of late-onset sporadic AD is of hitherto unknown etiologic origin. However, several risk factors have been identified that predispose an individual to develop AD, among them most prominently the epsilon4 allele of apolipoprotein E (ApoE) and the B-allele of cystatin C. The late onset and complex pathogenesis of neurodegenerative disorders pose a formidable challenge to the development of therapeutic agents.

Currently, there is no cure for AD, nor even a method to diagnose AD ante-mortem with high probability. However, β-amyloid has become a major target for the development of drugs designed to reduce its formation (Vassar, R. et al., Science 286, 735-41, 1999), or to activate mechanisms that accelerate its clearance from brain.

However, first experimental results by Schenk et al. (Nature, vol. 400, 173-177, 1999; Arch. Neurol., vol. 57, 934-936, 2000) suggest possible new treatment strategies for AD. The PDAPP transgenic mouse, which overexpresses mutant human APP (in which the amino acid at position 717 is phenylalanine instead of the normal valine), progressively develops many of the neuropathological hallmarks of AD an age- and brain region-dependent manner. Transgenic animals were immunised with Aβ₄₂ either before the onset of AD-type neuropathologies (at 6 weeks of age) or at an older age (11 months), when amyloid-β deposition and several of the subsequent neuropathological changes were well established. Immunisation of the young animals essentially prevented the development of β-amyloid-plaque formation, neuritic dystrophy and astrogliosis. Treatment of the older animals also markedly reduced the extent and progression of these AD-like neuropathologies. It was shown that Aβ₄₂ immunisation results in the generation of anti-Aβ antibodies and that Aβ-immunoreactive monocytic/microglial cells appear in the region of remaining plaques. However, an active immunisation approach can entail serious side effects and hitherto unknown complications in human subjects.

Bard et al. (Nature Medicine, Vol. 6, Number 8, 916-919, 2000) reports that peripheral administration of antibodies against amyloid β-peptide is sufficient to reduce amyloid burden. Despite their relatively modest serum levels, the passively administered antibodies were able to cross the blood-brain barrier and enter the central nervous system, decorate plaques and induce clearance of pre-existing amyloid. However, even a passive immunisation against βpeptide may cause undesirable side effects in human patients.

The present invention is directed to using recombinant protein to treat Alzheimer's patients. The balance between the anabolic and catabolic pathways in the metabolism of the Aβ peptides is delicate. Although considerable effort has focused on the generation of the Aβ peptides, until recently considerably less emphasis has been placed on the clearance of these peptides. Removal of extracellular Aβ peptide appears to proceed through two general mechanisms; cellular internalization and extracellular degradation. The invention describe a novel approach with will complement the natural catabolic process of amyloid β peptide.

DeMattos (PNAS 98: 8850-8855. 2001) have described the sink hypothesis that state that Aβ-peptide can be removed from CNS indirectly by lowering the concentration of the peptide in the plasma. They used an antibody that binds the Aβ-peptide in the plasma and thereby sequester Aβ from the CNS. This is accomplished because the antibody prevent influx of Aβ from the plasma to CNS and/or change the equilibrium between the plasma and CNS due to a lowering of the free Aβ concentration in plasma. Amyloid binding agents unrelated to antibodies have also been shown to be effective in removing amyloid β-peptide from CNS through the binding in plasma. Matsuoka et al. (J. Neuroscience, Vol. 23: 29-33, 2003) have presented data using two amyloid β-peptide binding agents, gelsolin and GM1, which sequester plasma Aβ and thereby reduce or prevent brain amyloidosis.

Another approach to remove or eliminate Aβ-peptide is the use of a degradation enzyme that degrades the amyloid β peptide into smaller fragments with no or lower toxicological effects which are more prone for clearance. This enzymatic digestion of the Aβ-peptide will also work through the sink hypothesis mechanism by lowering the free concentration of amyloid β peptide in plasma. However, there is also a possibility for direct clearance of amyloid β peptide in the CNS and/or CSF. This approach will not only lower the free concentration of Aβ but also directly clear the environment from the full-length peptide. This approach is advantageous because it will not increase the total (free and bound) concentration of Aβ in the plasma as been seen in cases when using amyloid β peptide binding agents such as antibodies. There are enzymes described in the literature that degrade the Aβ-peptide at multiple sites, for example NEP (Leissring et al., JBC. 278: 37314-37320, 2003). Degradation of the Aβ-peptide at multiple site will generate small fragment that are cleared from the blood stream easily.

### BRIEF DESCRIPTION OF THE DRAWINGS

FIG. 1 shows schematically the overall structure of the fusion protein described in this invention with an enzymatic or catalytic active amyloid β peptide degrading component and a half-life modulator component that primarily modulate the half-life of the fusion protein in plasma and also changes the stability of the fusion protein. A linker between the two different components is optional and can be designed to position the two components in an optimal geometry or just covalently or non-covalently connect them in a flexible manner. Thus, a direct linkage between the two components is an option because they can be linked directly to each other. Direct linkage/connection means that part of the amyloid β peptide degrading components are connected to the half-life modulator component.

FIG. 2 shows an in vitro experiment that describes the approach to possibly lower amyloid β peptide in one compartment by degrading the free form of amyloid β peptide in another compartment, separated by a membrane that allow free transfer of amyloid β peptide between the compartments. The two different compartments could be plasma and CNS and the membrane reflecting the blood-brain-barrier (BBB).

FIG. 3 shows the partitioning of the amyloid β peptide between the free form, plaque formation, BBB crossing, binding and degradation. Importantly, degradation of amyloid β peptide in plasma lowers the free form of amyloid β peptide in CNS and thereby prevents the formation of amyloid plaques. A similar effect is produced when using a binding agent.

FIG. 4 shows the main difference between binding and degradation of amyloid β peptide. When a binding approach is used, a potential built up of amyloid β peptide in plasma is possible which might have a negative effect in the peripheral system. The binding also competes with the natural process of catabolism of amyloid β peptide. On the other hand, the invention describing the fusion protein will directly degrade the amyloid β peptide in plasma and the degradation will complement the natural pathway of catabolism of amyloid β peptide.β

FIG. 5 shows the amino acid sequence of human amyloid beta A4 protein [Precursor]. The amyloid β peptide corresponding to amino acid 672 to 714 in the sequence (amino acid 1-43), DAEFRHDSG YEVHHQKLVF FAEDVGSNKG AIIGLMVGGV VIAT. Predominant forms of the amyloid β peptide also include any shorter forms of this peptide, especially 1-38, 1-40 and 1-42 but not restricted to these forms.

FIG. 6 shows the cleavage sites in the amyloid β peptide that have been proposed and describe in the literature. Neprilysin (NEP) cleavage sites are indicated by the letter c. There are 13 potential cleavage sites for NEP in the amyloid β peptide. Purified Neprilysin has been reported to cleave amyloid β peptide in vitro at five sites (Howell et al., 1995) and has a Kₘ of 2.8 mM for Ab₁₋₄₂ (Takaki et al., 2000).

FIG. 7 shows some possibilities to common used modifications to prolong the plasma half-life of molecules in plasma. The Fc part of an antibody, pegylation and glycosulation are the three most frequently used approaches, but the invention is not restricted to these approaches.

FIG. 8 shows the overall structure of an IgG antibody. The light chain (V_{L} and H_{L}) is linked to the heavy chain (V_{H}, C_{H}1, C_{H}2, and C_{H}3) through a disulfide bond (—S—S—). The Fc region is composed of the C_{H}2 and C_{H}3 domains. The IgG antibody is an example of a class of antibody described as useful for this invention to modulate the plasma half-life if the fusion protein, however, other classes of antibody could also be used.

FIG. 9 shows where the IgG class is divided into four subclasses (γ1, γ2, γ3, γ4). These subclasses have different activity in the Fc region effecting for example complement activation. In addition, the various immunoglobulins are divided in five classes (IgG, IgM, IgA, IgE, IgD).

FIG. 10 shows schematically how the plasma half-life and concentration change dependent on the designed the protein. A protein modified with pegylation has a longer half-life compare to a protein that has not been modified (e.g. a recombinant protein). The schematic example also shows a prolongation of the plasma half-life using an Fc part (Fc-fusion) as a modulator. In some instance, identical prolongation seen for an Fc-fusion protein of the plasma half-life can be accomplished using pegylation. Glycosylation can also be used to prolong the plasma half-life. These various technologies or approaches are well known in art.

FIG. 11 shows the structure of neprilysin, which is composed of a small intracellular part, a transmembrane region and an extracellular region. The extracellular region (amino acid 52-749) is preferably used as the modulator component, which is linked to the modulator component. The precise amino acid sequence region described as the extracellular region is varied dependent and can include additional amino acids into the membrane region and additional amino acids in the C-terminal region.

FIG. 12 shows an example of a fusion protein where the amyloid β peptide-degrading enzyme is neprilysin and the plasma half-life modulator is an Fc component. The extracellular part of neprilysin are linked directly to the N-terminal end of the Fc region which means that the C-terminal end of neprilysin is linked to the Fc compartment. This fusion protein can be expressed and purified for therapeutic usage.

FIG. 13 shows different possibilities to link together an amyloid β peptide-degrading component with an amyloid β binding component with a plasma half-life modulator.

FIG. 14 shows an example of a bifunctional fusion protein where the amyloid β peptide-degrading component is neprilysin, the amyloid β peptide-binding component is a Fab fragment and the plasma half-life modulator component is an Fc part.

FIG. 15 shows the strategy for constructing a gene encoding a fusion between the extra cellular domain of Neprilysin and the Fc domain of IgG4 (including the hinge region of IgG4). An overlapping PCR will merge Neprilysin and IgG4 Fc. The gene will be introduced into a pGEM cloning vector and DNA sequenced.

FIG. 16 shows the strategy for introducing the gene encoding the signal peptide. The blunt-end restriction enzyme will be used to fuse the 5 ′ end of Neprilysin with the 3′ end of the signal sequence. The complete fragment contains Gateway sequences and will be transferred to a Gateway donor plasmid to facilitate recloning to any expression vector.

FIG. 17 shows enzymatic activity (production described in example 11 and activity measured as described in Example 18) in 5 L Bioreactor. FIG. 17a shows the activity in the cell media and FIG. 17b shows the specific activity, after correction for enzyme concentration.

FIG. 18 shows the activity measurements of Neprilysin, extracted from cell media using a biotinylated Nep-specific antibody and Streptavidin sepharose as described in example 13.

FIG. 19 Left pane: Western blot showing Neprilysin-Fc expression after 7 days expression (last lane, Nep control). Right pane: Affinity purified protein Neprilysin-Fc (IgG4), purified with affinity chromatography, and eluted with low pH.

FIG. 20 shows the time-dependent degradation of Aβ₁₋₄₀. 5 μg/ml neprilysin was incubated with guinea pig plasma for 0-360 minutes at 37° C. Experimetal details described in example 19.

FIG. 21 shows the time-dependent degradation of Aβ₁₋₄₂. 5 μg/ml neprilysin was incubated with guinea pig plasma for 0-360 minutes at 37° C. Experimetal details described in example 19.

FIG. 22 shows the dose-dependent degradation of Aβ₄₀. 0-20 μg/ml neprilysin was incubated with guinea pig plasma for 210 minutes at 37° C. Experimetal details described in example 19.

FIG. 23 shows that the degradation by added Neprilysin of Aβ₄₀ is inhibited by addition of 10 microM Phosphoramidon in plasma. Experimetal details are described in example 19.

FIG. 24 shows degradation of Amyloid β 1-40 peptide by recombinant human Neprilysin in a buffer system. Experimetal details are described in Example 20.

FIG. 25 shows the purified IDE-Fc analysed on SDS-PAGE and Western blot. IDE-Fc was detected using an antibody specific for IDE in Western blot. Lane 1 and 2: Purified IDE-Fc, lane 3: soluble IDE (0.1 μg). Experimetal details are shown in Example 24.

FIG. 26 shows the enzymatic activity of the purified IDE-FC construct. Panel A, shows fractions containing IDE-Fc activity and; Panel B, shows a detailed view of the samples in circles in Panel A. Also included in panel B is a control without any enzyme added. The activity measurements are described in Example 24.

### DISCLOSURE OF THE INVENTION

The object of the present invention is to provide fusion proteins capable of degrading Aβ peptide. Accordingly, the present invention provides a fusion protein having the formula A-L-M capable of degrading Aβ peptide at one or more cleavage sites in its amino acid sequence, wherein A is a component that cleaves the Aβ peptide; M is a component that modulates the half-life in plasma; and L is a component that connects A and M.

In one aspect there is provided such a fusion protein, wherein L covalently connects A and M.

In another aspect there is provided such a fusion protein, wherein A is a protease. Said protease may be an improved protease.

In yet another aspect there is provided such a fusion protein, wherein A is scaffold protein.

In yet another aspect there is provided such a fusion protein, wherein A is human Neprilysin. In one embodiment of this aspect, said Neprilysin is extracellular Neprilysin. Said extracellular Neprilysin may comprise an amino acid sequence according to any one of SEQ ID NO. 1, 2, 3 or 4.

In yet another aspect there is provided such a fusion protein, wherein A is insulin degrading enzyme.

In yet another aspect there is provided such a fusion protein, wherein M is a Fc part of an antibody. Said M may be an Fc part from an IgG antibody.

In yet another aspect there is provided such a fusion protein, wherein M is either pegylation or glycosylation or both.

In yet another aspect there is provided such a fusion protein, wherein L is selected from a peptide with a suitable sequence, a chemical linker and a direct connection between A and M.

In yet another aspect there is provided such a fusion protein, wherein A is human Neprilysin; M is an Fc part from an IgG antibody; and L is a peptide. Said Neprilysin may be extracellular Neprilysin and may comprise an amino acid sequence according to any one of SEQ ID NO. 1, 2, 3 or 4.

In yet another embodiment said fusion protein fusion protein according to claim 1, comprise the amino acid sequence according to SEQ ID NO. 8.

In yet another aspect there is provided such a fusion protein, wherein the combination of component A and component M connected through component L possesses a longer half-life that component A alone.

In another embodiment of the present invention there is provided a fusion protein having the formula (B:A)-L-M capable of degrading Aβ peptide at one or more cleavage sites, wherein B is a component that binds the Aβ peptide, wherein A is a component that cleaves the Aβ peptide; M is a component that prolongs the half-life in plasma; and L is a component that connects B and A with M.

In one aspect of this embodiment there is provided such a fusion protein, wherein L covalently connects A and M.

In another aspect of this embodiment there is provided such a fusion protein, wherein B is a protein that binds the amyloid β peptide.

In another aspect of this embodiment there is provided such a fusion protein, wherein B is a designed synthesized structure that binds amyloid β peptide

In another aspect of this embodiment there is provided such a fusion protein, wherein B is a part from an antibody that contain the complement determining regions (CDR) such as for example a Fab, scFv or single domain. Camel antibodies with only a heavy chain can be used a binding component.

In another aspect of this embodiment there is provided such a fusion protein, wherein B a scaffold protein that binds amyloid β peptide. Examples of scaffold proteins are tendamistat, affibody, anticalin and ankyrin.

In yet another aspect of this embodiment there is provided such a fusion protein, wherein A is selected from a protease, an improved protease and a scaffold protein.

In yet another aspect of this embodiment there is provided such a fusion protein, wherein A is human Neprilysin. Said Neprilysin may be extracellular Neprilysin and may comprise an amino acid sequence according to any one of SEQ ID NO. 1, 2, 3 or 4.

In yet another aspect of this embodiment there is provided such a fusion protein, wherein A is insulin degrading enzyme.

In yet another aspect of this embodiment there is provided such a fusion protein, wherein M is a Fc part of an antibody. M may be an Fc part from an IgG antibody

In yet another aspect of this embodiment there is provided such a fusion protein, wherein M is either pegylation or glycosylation or both.

In yet another aspect of this embodiment there is provided such a fusion protein, wherein L is selected from a peptide, a chemical linker and a direct connection between A and M.

In yet another aspect of this embodiment there is provided such a fusion protein, wherein A is human Neprilysin; B is a Fab fragment; M is an Fc part from an IgG antibody; and L is a peptide. Said Neprilysin may be extracellular Neprilysin and may comprise an amino acid sequence according to any one of SEQ ID NO. 1, 2, 3 or 4.

In yet another aspect of this embodiment there is provided such a fusion protein, wherein the combination of component A, component B and component M connected through component L possesses a longer half-life that component A alone or component B alone or component A and B connected together.

In yet another embodiment of the present invention there is provided a fusion protein having the formula A-L-M-L-B capable of degrading Aβ peptide at one or more cleavage sites, wherein B is a component that binds the Aβ peptide, wherein A is a component that cleaves the Aβ peptide; M is a part that prolongs the half-life in plasma; and L is a component that connects A with M and M with B.

In one aspect of this embodiment there is provided such a fusion protein, wherein L covalently connects A and M.

In another aspect of this embodiment there is provided such a fusion protein, wherein wherein A is selected from a protease, an improved protease and a scaffold protein.

In yet another aspect of this embodiment there is provided such a fusion protein, wherein A is human Neprilysin. Said Neprilysin may be extracellular Neprilysin and may comprise an amino acid sequence according to any one of SEQ ID NO. 1, 2, 3 or 4.

In yet another aspect of this embodiment there is provided such a fusion protein, wherein A is insulin degrading enzyme.

In yet another aspect of this embodiment there is provided such a fusion protein, wherein B is a protein that binds the amyloid β peptide.

In another aspect of this embodiment there is provided such a fusion protein, wherein B is a designed synthesized structure that binds the amyloid β peptide

In another aspect of this embodiment there is provided such a fusion protein, wherein B is a part from an antibody that contain the complement determining regions (CDR) such as for example a Fab, scFv or single domain. Camel antibodies with only a heavy chain can be used a binding component.

In another aspect of this embodiment there is provided such a fusion protein, wherein B a scaffold protein that binds amyloid β peptide. Examples of scaffold proteins are tendamistat, affibody, anticalin and ankyrin.

In yet another aspect of this embodiment there is provided such a fusion protein, wherein M is an Fc part of an antibody. M may be an Fc part from an IgG antibody.

In yet another aspect of this embodiment there is provided such a fusion protein, wherein M is selected from pegylation and glycosylation.

In yet another aspect of this embodiment there is provided such a fusion protein, wherein L is selected from a peptide, a chemical linker and a direct connection between A and M.

In yet another aspect of this embodiment there is provided such a fusion protein, wherein A is human Neprilysin; B is a Fab fragment; M is an Fc part from an IgG antibody; and L is a peptide. Said Neprilysin may be extracellular Neprilysin and may comprise an amino acid sequence according to any one of SEQ ID NO. 1, 2, 3 or 4.

In yet another aspect of this embodiment there is provided such a fusion protein, wherein the combination of component A, component B and component M connected through component L possesses a longer half-life that component A alone or component B alone or component A and B connected together.

The terms used throughout this specification are defined as follows, unless otherwise limited in specific instances.

The term “modulator” refers to a molecule that prevents degradation and/or increases plasma half-life, reduces toxicity, reduces immunogenicity, or increases biological activity of a therapeutic protein. Exemplary modulators include an Fc domain as well as a linear polymer (e.g., polyethylene glycol (PEG), polylysine, dextran, etc.); a branched-chain polymer (see, for example, U.S. Pat. No. 4,289,872, U.S. Pat. No. 5,229,490; WO 93/21259); a lipid; a cholesterol group (such as a steroid); a carbohydrate or oligosaccharide; or any natural or synthetic protein, polypeptide or peptide that binds to a salvage receptor. Glycosylation is also an example of modulator that through the increase in size of the fusion protein can prolong the plasma half-life, mainly due to a change in the clearance mechanism.

The term “protein” refers to a molecule that possesses a catalytic activity, which degrades the amyloid β peptide by protolytic cleavage at any possible site in the amino acid sequence. Examples of proteins include the neprilysin enzyme as well as other catalytic active enzymes that degrade the amyloid β peptide. Catalytic antibodies could also be used as the protein part. The protein can be a natural occurring variant from any species (e.g. human, monkey, mice) or a designed variant using rational design or molecular evolution technologies. The protein molecule can also be different polymorphic or splice variants.

The term “fusion” refers to a molecule that is composed of a modulator molecule and a protein molecule. The modulator may be covalently linked to the protein part to create the fusion protein. A non-covalent approach can also be used to connect the protein to the modulator part.

The term “degrade” or “degradation” refers to a process where one starting molecule is divided in two or more molecule(s). More specifically, the amyloid β peptide (in any size from amino acid 1-43 and smaller) is cleaved to generate smaller fragments compared to the starting molecule. The cleavage can be accomplished through hydrolysis of peptide bonds or other type of reaction, which split the molecule in smaller parts.

The term “native Fc” refers to molecule or sequence comprising the sequence of a non-antigen-binding fragment resulting from digestion of whole antibody, whether in monomeric or multimeric form. The original immunoglobulin source of the native Fc may be of human origin and may be any of the immunoglobulins, although IgG1 and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “Fc variant” refers to a molecule or sequence that is modified from a native Fc but still comprises a binding site for the salvage receptor, FcRn. Publications WO 97/34631 and WO 96/32478 describe exemplary Fc variants, as well as interaction with the salvage receptor, and are hereby incorporated by reference. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by other means.

The term “pharmacologically active” means that a substance so described is determined to have activity that affects a medical parameter (e.g., blood pressure, blood cell count, cholesterol level) or disease state (e.g., cancer, autoimmune disorders, dementia).

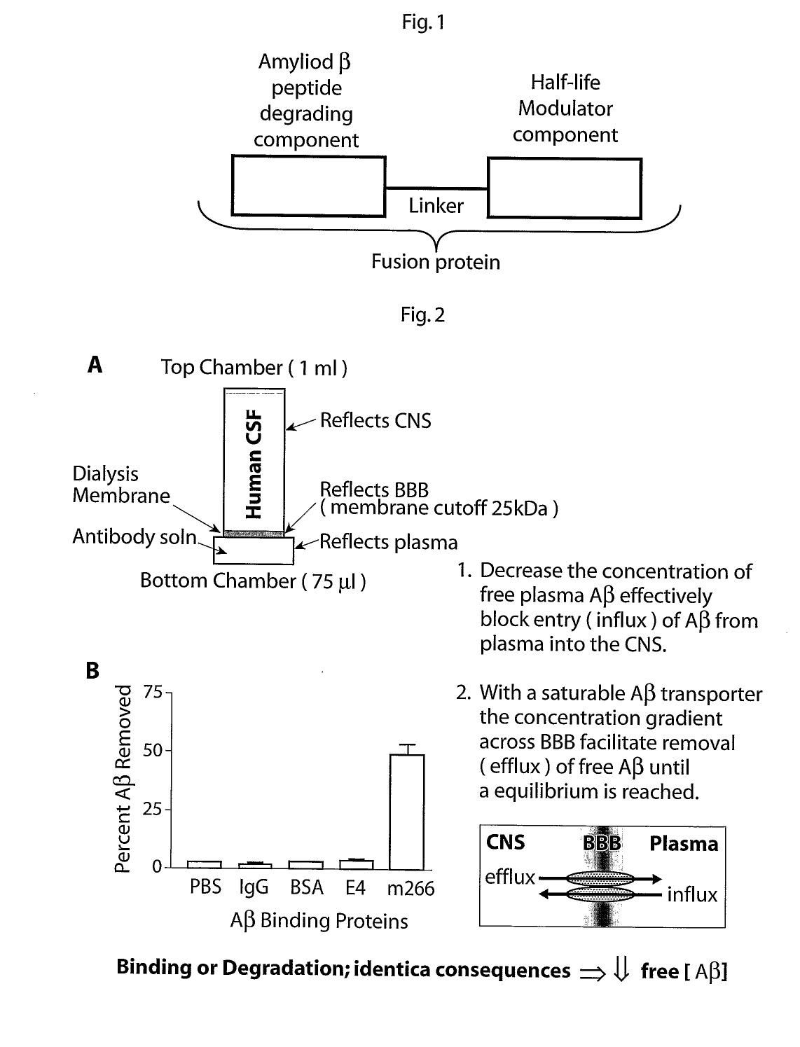

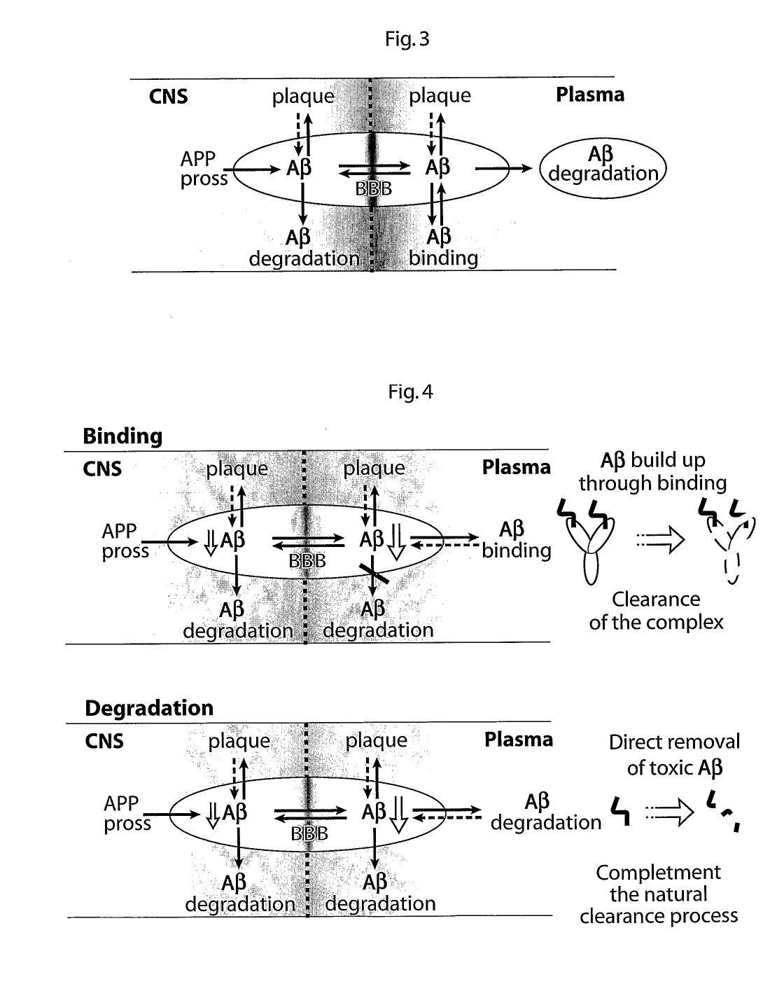

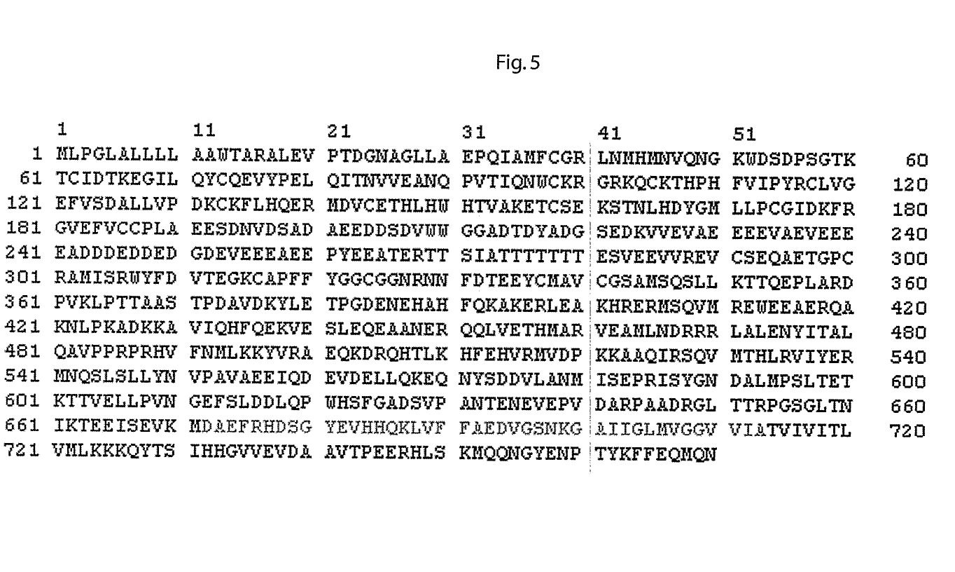

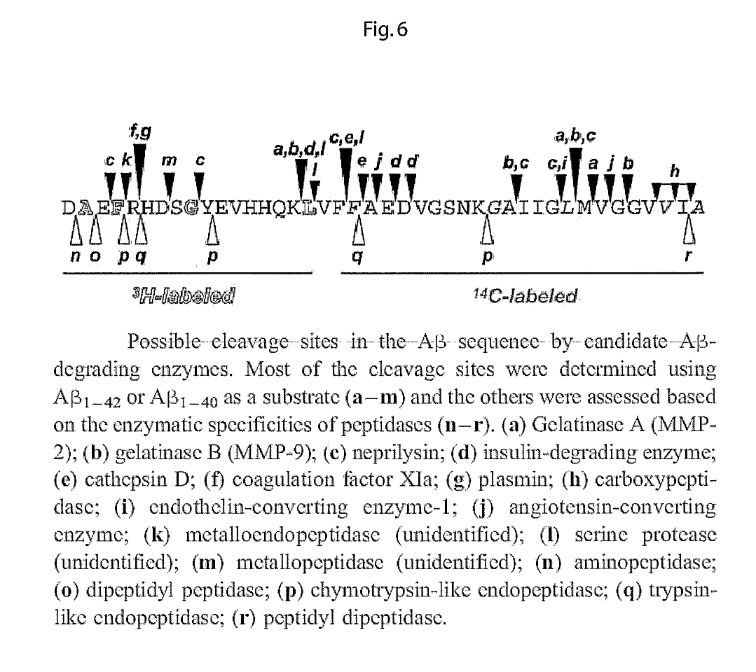

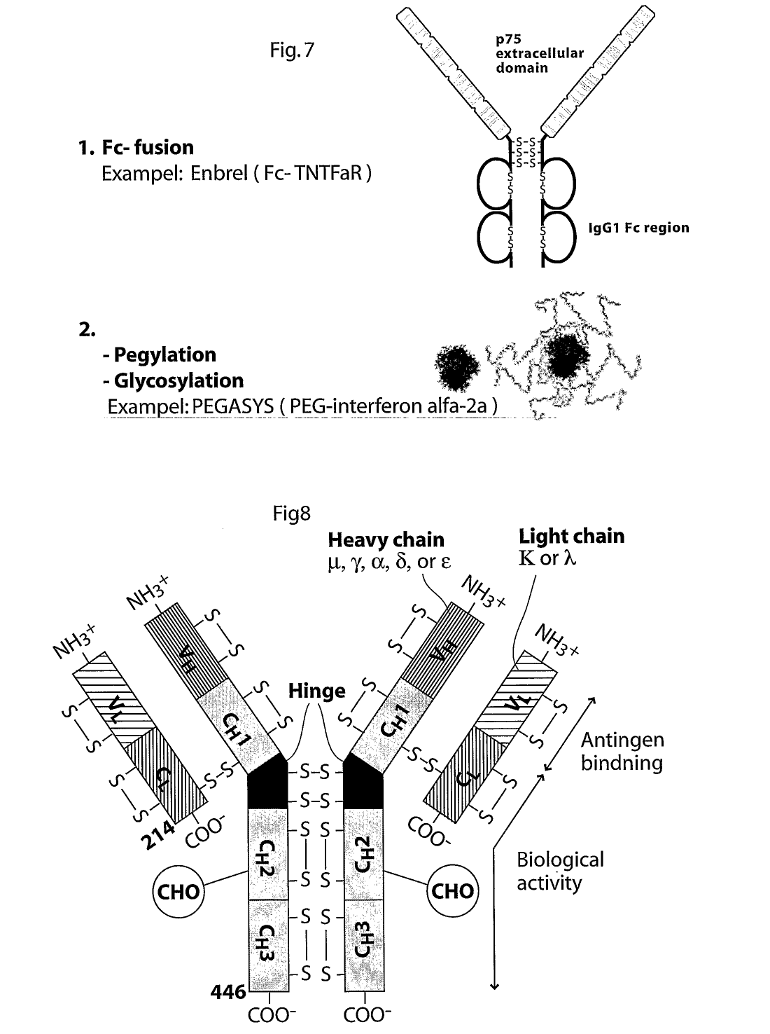

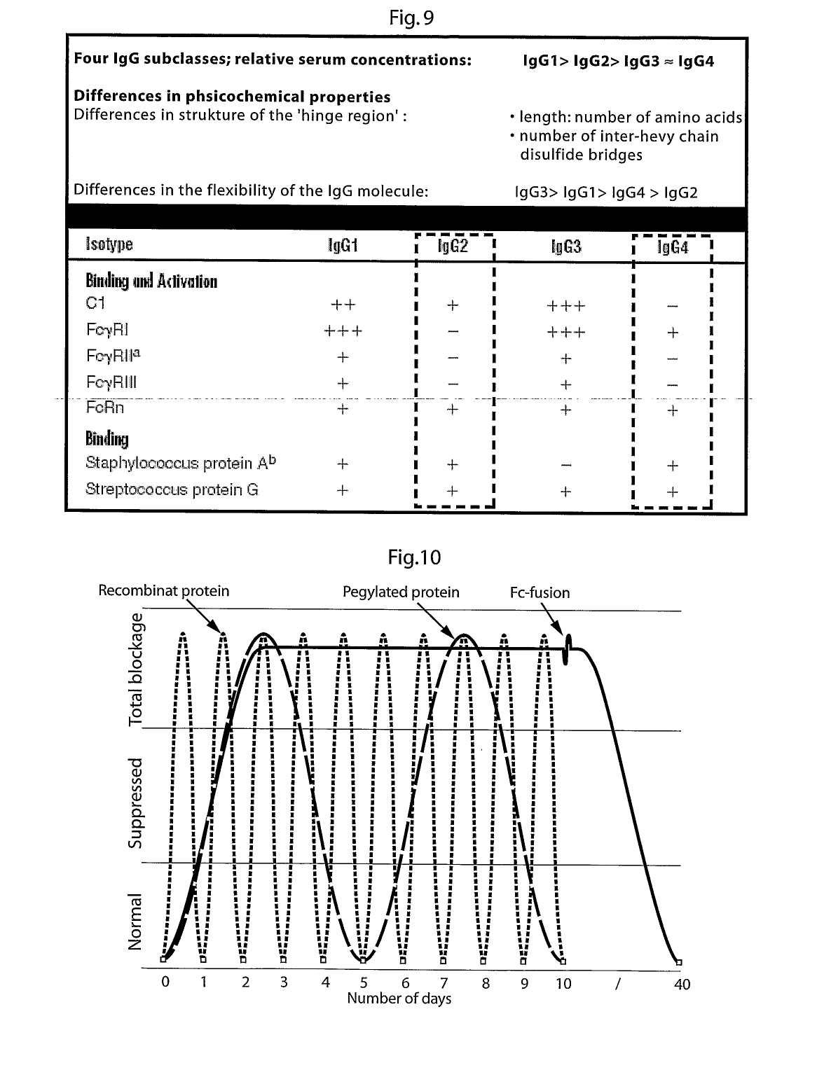

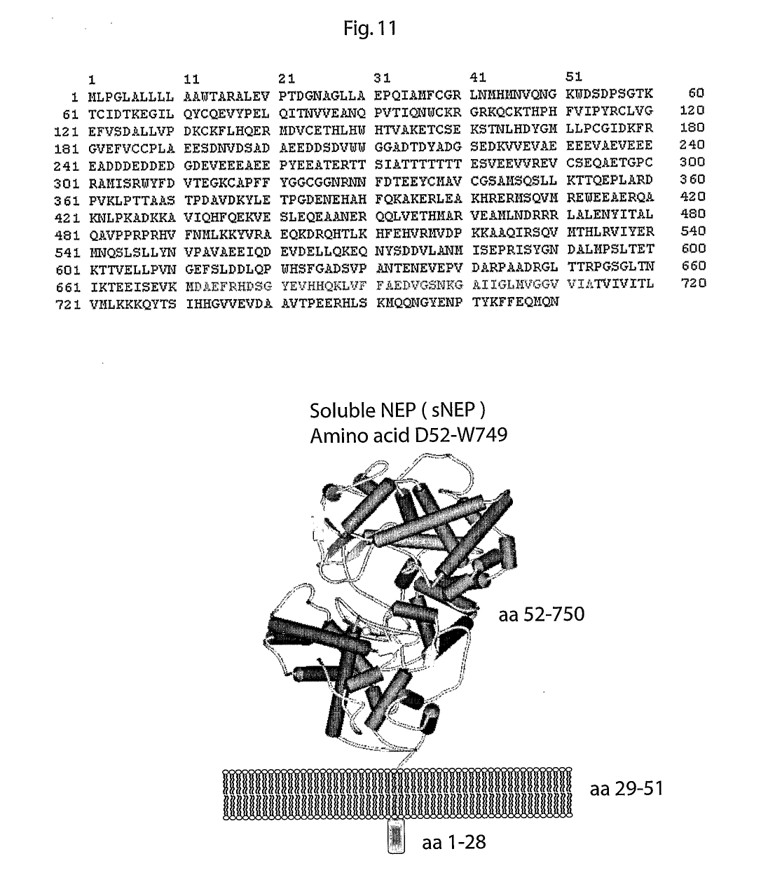

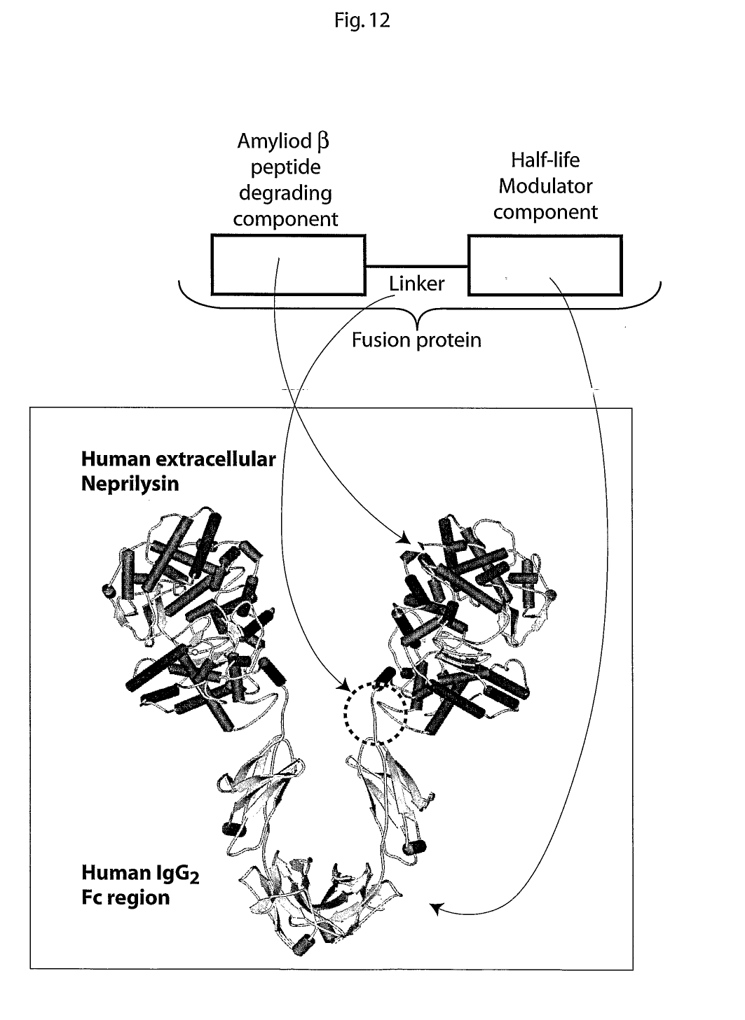

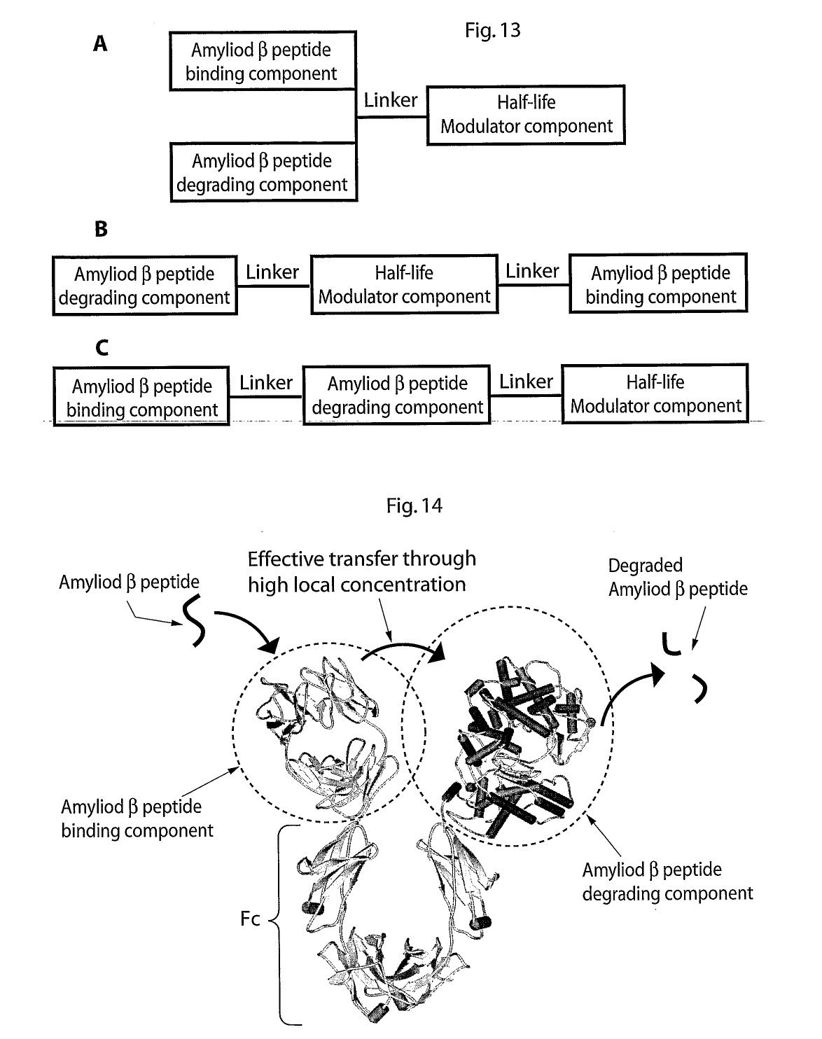

The term “amyloid beta peptide”, “Aβ peptide” or “amyloid β peptide” means any form of the peptide that correlate to amino acid sequpeptideence (one letter code) DAEFRHDSG YEVHHQKLVF FAEDVGSNKG AIIGLMVGGV VIAT (SEQ ID NO 50) in the human Aβ A4 protein [Precursor], corresponding to amino acid 672 to 714 in the sequence (amino acid 1-43). It also includes any shorter forms of this peptide, such as 1-38, 1-40 and 1-42 but not restricted to these forms. Moreover, Amyloid β peptide has several natural occurring forms. The human forms of Amyloid β peptide are referred to as Aβ39, Aβ40, Aβ41, Aβ42 and Aβ43. The sequences of these peptides and their relationship to the APP precursor are illustrated by FIG. 1 of Hardy et al., TINS 20, 155-158 (1997). For example, Aβ42 has the sequence:

H2N-Asp-Ala-Glu-Phe-Arg-His-Asp-Ser-Gly-Tyr-Glu-Val-His-His-Gln-Lys-Leu-Val-Phe-Phe-Ala-Glu-Asp-Val-Gly-Ser-Asn-Lys-Gly-Ala-Ile-Ile-Gly-Leu-Met-Val-Gly-Gly-Val-Val-IIe-Ala-OH (SEQ ID NO 51). Aβ41, Aβ40 and Aβ39 differ from Aβ42 by the omission of Ala, Ala-Ile, and Ala-Ile-Val respectively from the C-terminal end. Aβ43 differs from Aβ42 by the presence of a threonine residue at the C-terminus. Overall, amyloid beta peptide means the peptide form that is involved in plaque formation that cause alzhiemer disease.

The term “half-life” is defined by the time taken for the removal of half the initial concentration of the fusion protein from the plasma. This invention describes ways of modulating the half-life in plasma. Such modification can produce fusion proteins with improved pharmacokinetic properties (e.g., increased in vivo serum half-life). Prolong the half-life means that it takes longer time to remove or get a clearance of half of the initial concentration of the fusion protein from the plasma. Half-life of a pharmaceutical or chemical compound is well defined and known in the art.

The term “connect” means a covalent or a reversible linkage between two or more parts. A covalent linkage can for example be a peptide bond, disulfide bond, carbon-carbon coupling or any type of linkage that is based of a covalent linkage between to atoms. Reversible linkage can for example be biotin-streptavidin, antibody-antigen or a linkage, which is classified as a reversible linkage known in the art. For example, a covalent linkage is directly obtained when the protein part and the modulator part of the fusion protein is produced in a recombinant form from the same plasmid, thus the connection is designed on DNA level.

The term “cleavage sites” means a specific location/site in a peptide sequence that can be cleaved by a protein or an enzyme. Cleavage is normally produced by hydrolysis of the peptide bond connecting two amino acids. Cleavage can also take place at multiple sites in the same peptide using a single or a combination of proteins or enzymes. A cleavage site can also be other site than the peptide bond. This invention describes the cleavage of the amyloid β peptide in detail.

The term “binding domain” means a molecule that binds the amyloid β peptide with an affinity of that is therapeutically relevant. These molecules bind to amyloid β peptide with a binding affinity greater than or equal to about 10⁶, 10⁷, 10⁸, 10⁹, or 10¹⁰M⁻¹. Typical binding domains are, but not restricted to, antibodies (e.g. Fab, scFv, single domains all including the CDR regions), scaffold proteins as described in this invention and in the literature or synthetically produced molecules with affinity for the amyloid β peptide.

The term “protease” means any protein molecule acting in the hydrolysis of peptide bonds. It includes naturally occurring proteolytic enzymes, as well as variants thereof obtained by site-directed or random mutagenesis or any other protein engineering method, any fragment of an proteolytic enzyme, or any molecular complex or fusion protein comprising one of the aforementioned proteins. The protease can be a serine, cysteine, aspartic or a metalloprotease.

The term “substrate” or “peptide substrate” means any peptide, oligopeptide, or protein molecule of any amino acid composition, sequence or length, that contains a peptide bond that can be hydrolyzed catalytically by a protease. The peptide bond that is hydrolyzed is referred to as the “cleavage site”. Numbering of positions in the substrate is done according to the system Introduced by Schlechter & Berger (Biochem. Biophys. Res. Commun. 27 (1967) 157-162). Amino acid residues adjacent N-terminal to the cleavage site are numbered P1, P2, P3, etc., whereas residues adjacent C-terminal to the cleavage site are numbered P1′, P2′, P3′, etc. The substrate or peptide substrate of this invention is the amyloid β peptide.

The term “specificity” means the ability of a protein or a protease to recognize and hydrolyze selectively certain peptide substrates while others remain uncleaved. Specificity can be expressed qualitatively and quantitatively. “Qualitative specificity” refers to the kind of amino acid residues that are accepted by a protease at certain positions of the peptide substrate. Proteases that accept only a small portion of all possible peptide substrates have a “high specificity”. Proteases that accept almost any peptide substrate have a “low specificity”. Proteases with very low specificity are also referred to as “unspecific proteases”.

The term “evolved protease” describes any protease that have been obtained using random PCR, DNA shuffling or other type of methods that generate diversity on the DNA/RNA level. Literature describing these approaches is for example; D. A. Drummond, B. L. Iverson, G. Georgiou and F. H. Arnold, Journal of Molecular Biology 350: 806-816 (2005) and S. McQ and D.S. Tawfik, Biochemistry 44: 5444-5452 (2005).

The term “improved protease” describes any protease variants that possess higher catalytic activity if that is needed. However, in some instances a lower catalytic activity might be preferable. Improved protease might also mean a variant which cleaves a certain substrate compare to another substrate more efficient that that the original protease. Improved means a more preferred property, such as catalytic activity and/or selectivity to obtain a more is optimized pharmaceutic compound.

The term “human Neprilysin” refers to any natural form of human neprilysin. This includes all splice and polymorphic variants that naturally occur in the human population. A number of forms of human neprilysin is described in this invention (SEQ ID Nos 1 to 4).

The term “scaffold protein” describes any protein that binds amyloid β peptide. Examples of scaffold proteins are tendamistat, affibody, anticalin and ankyrin. These scaffold proteins are typically designed and is based on a rigid core structure and a part, loops, surfaces or cavities that can be randomized for the identification of binders. These scaffold proteins are well described in the literature.

This invention suggests the possibility that the administration of an optimized recombinant Aβ degradation enzyme inhibits amyloid plaque formation by decreasing brain levels of Aβ. As a consequence, amyloid plaque-related astrogliosis will also be reduced.

In one aspect of this invention the therapeutic compound is of fully human origin. The fusion protein is composed of fully human proteins that are linked together using a linker with lowest possible immunogenic activity.

Advantages using a degrading enzyme compared to a binding molecule such an antibody are:
Degradation with an enzyme of the amyloid β peptide will directly remove the toxic effect compare to a binding approach where the concentration of the amyloid β peptide could potentially increase if the binding molecule in complex with amyloid β peptide is not cleared fast enough. This could be harmful especially if the amyloid β peptide concentration increases peripherally.
Catalytic degradation of amyloid β peptide will remove the peptide more efficiently that binding. Only a catalytic amount of the degrading enzyme will be necessary to remove sufficient amyloid β peptide whereas a binding molecule such as an antibody, a stoichiometric amount will be needed for a therapeutic effect. This will have a great impact on the amount needed for therapeutic treatment.
If the binding molecule is an antibody and cross the BBB allowing binding to the amyloid β peptide in the plaques, a potential immunological respons that are harmful is possible. On the other hand, a catalytic fusion protein will not bind to the plaques and use the Fc reactivity but only reduce the free concentration of amyloid β peptide. Thus, A catalytic enzyme will only degrade the free pool of amyloid β peptide. A binding agent like an antibody could potentially enter the CNS and dissolve the plaques through Fc activity. This might be unfavorable if large amount of amyloid β peptide is released in the vicinity of the plaque and they are toxic to the cells.

One important enzyme in Aβ catabolism is Neprilysin, also known as neutral endopeptidase-24.11 or NEP. Iwata et al. (Nature Medicine, 6: 143-149, 2000) showed that the Aβ₁₋₄₂ peptide underwent full degradation through limited proteolysis conducted by NEP similar or identical to neprilysin as biochemically analysed. Consistently, NEP inhibitor infusion resulted in both biochemical and pathological deposition of endogeneous Aβ₄₂ in brain. It was found that this NEP-catalysed proteolysis therefore limits the rate of Aβ42 catabolism.

NEP is a 94 kD, type two membrane-bound Zn-metallopeptidase implicated in the inactivation of several biologically active peptides including enkephalins, tachykinins, bradykinin, endothelins and atrial natriuretic peptide. NEP is present in peptidergic neurons in the CNS, and its expression in brain is regulated in a cell-specific manner (Roques B. P. et al., Pharmacol. Rev. 45, 87-146, 1993; Lu B. et al., J. Exp. Med. 181, 2271-2275, 1995; Lu B. et al., Ann. N.Y. Acad. Sci. 780, 156-163, 1996). While type 2 NEP-transcripts are absent from the CNS, type 1 and type 3 transcripts are localized in neurons and in oligodendrocytes of the corpus callosum, respectively (Li C. et al., J. Biol. Chem. 270, 5723-5728, 1995). The Neprilysin family of proteases and endopeptidases comprises structurally or functionally homologous members of NEP such as the recently described NEP II gene and its isoforms (Ouimet T. et al., Biochem. Biophys. Res. Commun. 271:565-570, 2000), which are expressed in the CNS in a complementary pattern to NEP. A further member of this family is NL-1 (neprilysin like 1), a soluble protein efficiently inhibited by the NEP inhibitor phosphoramidon (Ghaddar G. et al., Biochem. J. 347: 419-429, 2000).

Other enzymes that are known to catabolism Aβ have also been described. The zinc metallopeptidase insulin-degrading enzyme (IDE, EC. 3.4.22.11) cleaves Aβ₁₋₄₀ and Aβ₁₋₄₂ into what appears to be innocuous products. IDE is a true peptidase; it does not hydrolyze proteins. The enzyme cleaves a limited number of peptides in vitro including insulin and insulin related peptides, β endorphin, and Aβ peptides. IDE has been suggested to be one of the physiological Aβ metabolizing enzymes (W. Q. Qui et al. (1998) J. Biol. Chem. 273, 32730-32738). Kurichkin and Goto (I. V. Kurochkin and S. Gato (1994) FEBS Lett. 345, 33-37) first reported that insulin degrading enzyme can hydrolyze Aβ₁₋₄₀. This finding was confirmed in two separate studies (W. Q. Qui et al. (1998) J. Biol. Chem. 273, 32730-32738; and J. R. McDermott and A. M. Gibson (1997) Neurochem. Res. 22, 49-56). Moreover, metalloprotease 24.15, a recently identified as a Aβ-degrading enzyme (Yamin R. et al., J. Biol. Chem. 274, 18777-18784, 1999), was also unchanged in response to Aβ injections. Angiotensin converting enzyme (ACE), an unrelated neuronal Zn-metalloendo peptidase have been also mention as a possible Aβ-peptide degrading enzyme (Barnes N. M. et al., Eur. J. Pharmacol. 200, 289-292,1991; Alvarez R. et al., J. Neurol. Neurosurg. Psychiatry 67, 733-736, 1999; Amouyel P. et al., Ann. N.Y. Acad. Sci. 903, 437-441, 2000) with no known affinity to Aβ (McDermott J. R. and Gibson A. M., Neurochem. Res. 22, 49-56, 1997).

The sequence used from the neprilysin may be the extracellular part of the protein. The extracellular part is defined as the part of neprilysin that is defined as outside the membrane region. This invention also includes the use of the whole sequence of neprilysin as the amyloid β peptide-degrading component. The invention also comprises smaller fragments of neprilysin as long as the catalytic activity-is preserved against the amyloid β peptide. The invention also comprises any polymorphism variants and splice variants of neprilysin.

This invention describes a novel and alternative strategy to hydrolyze Aβ peptides before they form amyloid plaques or at least prevent the further development of existing plaques. It may also be possible to remove existing plaques by hydrolyzing any plaque-derived Aβ peptide in equilibrium with free Aβ peptide.

Another embodiment of the present invention refers to a molecule that is composed of one part that binds amyloid β peptide with high affinity. This affinity is below micromolar in binding affinity. The binding affinity for amyloid β peptide is preferably at nanomolar in binding affinity. The other part that is involved in the interaction with amyloid β peptide is an active component that cleaves the amyloid β peptide at one or more site in the structure of the amyloid β peptide. The reason to combine a binding part linked together with a catalytic active part that both recognize the amyloid β peptide is that the binding part binds the amyloid β peptide and thereby increase the local concentration (the binding part and the catalytic part) is binding to the dissociated form of amyloid β peptide. Some bind specifically to the dissociated form without binding to the aggregated form. Some bind to both aggregated and dissociated forms. Some such antibodies bind to a naturally occurring short form of Aβ (i.e covalently or in another way linked together) of amyloid β peptide to become cleaved by the active part that is locally around due to the linkage engineered in the bifunctional molecule. The linkage between the amyloid β peptide binding component and the amyloid β peptide-degrading component is preferably mediated by the plasma half-life modulator component with or without a linker component.

In some embodiments of this invention the therapeutic agents include fusion proteins that specifically bind to amyloid β peptide or other component of amyloid plaques. Such compound can be a part of a monoclonal or polyclonal or any other amyloid β peptide binding agent. These compounds bind to amyloid β peptide with a binding affinity greater than or equal to about 10⁶, 10⁷, 10⁸, 10⁹, or 10¹⁰M⁻¹. These binding components are preferably connected with an amyloid β peptide-degrading component.

One aspect of the invention refers to the combination with the “Fc” domain of an antibody with a amyloid β peptide degrading component in the fusion protein. Antibodies comprise two functionally independent parts, a variable domain known as “Fab”, which binds antigen, and a constant domain known as “Fc”, which links to such effector functions as complement activation and attack by phagocytic cells. An Fc has a long serum half-life, whereas a Fab is short-lived. Capon et al. (1989), Nature 337: 525-31. When constructed together with a therapeutic protein, an Fc domain can provide longer half-life or incorporate such functions as Fc receptor binding, protein A binding, complement fixation and perhaps even placental transfer.

Preferred molecules in accordance with this invention are Fc-linked amyloid α peptide degrading protein such as NEP-related proteins.

Useful modifications of protein therapeutic agents by fusion with the Fc domain of an antibody are discussed in detail in a publication entitled, “Modified Peptides as Therapeutic Agents,” WO 99/25044. That publication discusses linkage to a “vehicle” such as PEG, dextran, or an Fc region.

IgG molecules interact with three classes of Fc receptors (FcR) specific for the IgG class of antibody, namely FcγRI, FcγRII and FcγRIII. In preferred embodiments, the immunoglobulin (Ig) component of the fusion protein has at least a portion of the constant region of an IgG that has a low binding affinity for at least one of FcγRI, FcγRII or FcγRIII. In one aspect of the invention, the binding affinity of fusion proteins for Fc receptors is reduced by using heavy chain isotypes as fusion partners that have reduced binding affinity for Fc receptors on cells. For example, both human IgG1 and IgG3 have been reported to bind to FcRγI with high affinity, while IgG4 binds 10-fold less well, and IgG2 does not bind at all. The important sequences for the binding of IgG to the Fc receptors have been reported to be located in the CH2 domain. Thus, in a preferred embodiment, an antibody-based fusion protein with enhanced in vivo circulating half-life is obtained by linking at least the CH2 domain of IgG2 or IgG4 to a second non-immunoglobulin protein. For example, of the four known IgG isotypes, IgG1 (Cγ1) and IgG3 (Cγ3) are known to bind FcRγI with high affinity, whereas IgG4 (Cγ4) has a 10-fold lower binding affinity, and IgG2 (Cγ2) does not bind to FcRγI.

In one embodiment, the Aβ-peptide degrading component of the fusion protein is an enzyme. The term “enzyme” is used herein to describe proteins, analogs thereof, and fragments thereof which are active as proteases or petidases. Preferably, enzymes include serine, aspartic, metallo and cysteine proteases. Preferably, the fusion protein of the present invention displays enzymatic biological activity.

In another embodiment, the immunoglobulin domain is selected from the group consisting of the Fc domain of IgG, the heavy chain of IgG, and the light chain of IgG. In another embodiment, the constant region of the antibody in the fusion protein will be of human origin, and belong to the immunoglobulin family derived from the IgG class of immunoglobulins, in particular from classes IgG1, IgG2, IgG3 or IgG4, preferably from the class IgG2 or IgG4. It is also alternatively possible to use constant regions of immunoglobulins belonging to the IgG class from other mammals, in particular from rodents or primates; however, it is also possible, according to the invention, to use constant regions of the immunoglobulin classes IgD, IgM, IgA or IgE. Typically, the antibody fragments that are present in the construct according to the invention will comprise the Fc domain CH₃, or parts thereof, and at least one part segment of the Fc domain CH₂. Alternatively, it is also possible to conceive of fusion constructs according to the invention which contain, as component (A), the CH₃ domain and the hinge region, for the dimerization.

However, it is also possible to use derivatives of the immunoglobulin sequences which are found in the native state, in particular those variants which contain at least one replacement, deletion and/or insertion (combined here under the term “variant”). Typically, such variants possess at least 90%, preferably at least 95%, and more preferably at least 98%, sequence identity with the native sequence. Variants, which are particularly preferred in this context, are replacement variants which typically contain less than 10, preferably less than 5, and very particularly preferably less than 3, replacements as compared with the respective native sequence. Attention is drawn to the following replacement possibilities as being preferred: Trp with Met, Val, Leu, Ile, Phe, His or Tyr, or vice versa; Ala with Ser, Thr, Gly, Val, Ile or Leu, or vice versa; Glu with Gln, Asp or Asn, or vice versa; Asp with Glu, Gln or Asn, or vice versa; Arg with Lys, or vice versa; Ser with Thr, Ala, Val or Cys, or vice versa; Tyr with His, Phe or Trp, or vice versa; Gly or Pro with one of the other 19 native amino acids, or vice versa.

Soluble receptor-IgG fusion proteins are common immunological reagents and methods for their construction are known in the art (see e.g., U.S. Pat. No. 5,225,538). A functional amyloid β peptide-degrading domain may be fused to an immunoglobulin Fc domain derived from an immunoglobulin class or subclass. The Fc domains of antibodies belonging to different Ig classes or subclasses can activate diverse secondary effector functions. Activation occurs when the Fc domain is bound by a cognate Fc receptor. Secondary effector functions include the ability to activate the complement system, to cross the placenta, and to bind various microbial proteins. The properties of the different classes and subclasses of immunoglobulins are described in Roitt et al., Immunology, p. 4.8 (Mosby-Year Book Europe Ltd., 3d ed. 1993). The Fc domains of antigen-bound IgG1, IgG3 and IgM antibodies can activate the complement enzyme cascade. The Fc domain of IgG2 appears to be less effective, and the Fc domains of IgG4, IgA, IgD and IgE are ineffective at activating complement. Thus one can select an Fc domain based on whether its associated secondary effector functions are desirable for the particular immune response or disease being treated with the amyloid β peptide degrading-Fc fusion protein. If it would be advantageous to harm or kill target cells, one could select an especially active Fc domain (IgG1) to make the amyloid β peptide degrading-Fc-fusion protein. Alternatively, if it would be desirable to produce the amyloid β peptide degrading-Fc-Fusion without triggering the complement system, an inactive IgG4 Fc domain could be selected. This invention describes a fusion protein with a catalytic component linked to a Fc part and not a direct binding component. This means that the effect and activity from the Fc will be limited because many Fc effects are mediated through the binding. For example complement activation is dependent on binding and the formation of a network.

C-terminally of the immunoglobulin fragment, a fusion construct according to the invention typically, but not necessarily, contains a transition region between catalytic and modulator part, which transition region can in turn contain a linker sequence, with this linker sequence preferably being a peptide sequence. This peptide sequence can have a length from between 1 and up to 70 amino acids, where appropriate even more amino acids, preferably from 10 to 50 amino acids, and particularly preferably between 12 and 30 amino acids. The linker region of the transition sequence can be flanked by further short peptide sequences which can, for example, correspond to DNA restriction cleavage sites. Any restriction cleavage sites with which the skilled person is familiar from molecular biology can be used in this connection. Suitable linker sequences are preferably artificial sequences which contain a high number of proline residues (for example at every second position in the linker region) and, in addition to that, preferably have an overall hydrophilic character. A linker sequence, which consists of at least 30% of proline residues, is preferred. The hydrophilic character can preferably be achieved by means of at least one amino acid having a positive charge, for example lysine or arginine, or negative charge, for example aspartate or glutamate. Overall, the linker region therefore also preferably contains a high number of glycine and/or proline residues in order to confer on the linker region the requisite flexibility and/or rigidity.

However, native sequences, for example those fragments of ligands belonging to the NEP family which are disposed extracellularly, but immediately act, i.e. in front of, the cell membrane, are also suitable for use as linkers, where appropriate after replacement, deletion or insertion of the native segments as well. These fragments are preferably the 50 AA which follow extracellularly after the transmembrane region or else subfragments of these first 50 AA. However, preference is given to these segments having at least 85% sequence identity with the corresponding natural human sequences, with very particular preference being given to at least 95% sequence identity and particular preference being given to at least 99% sequence identity in order to limit the immunogenicity of these linker regions in the fusion protein according to the invention and not elicit any intrinsic humoral defense reaction. Within the context of the present invention, the linker region should preferably not possess any immunogenicity.

However, as an alternative to peptide sequences which are linked to the amyloid β peptide degrading component and the plasma half-life modulator component, by way of amide-like bonds, it is also possible to use compounds which are of a nonpeptide or pseudopeptide nature or are based on noncovalent bonds. Examples which may be mentioned in this connection are, in particular, N-hydroxysuccinimide esters and heterobifunctional linkers, such as N-succinimidyl-3-(2-pyridyldi-thio) propionate (SPDP) or similar crosslinkers.

Other ways of regulating the plasma half-life is to use pegylation or other type of modifications that increasing the molecular weight such as glycosylation.

As noted above, polymer modulators may also be used. Various means for attaching chemical moieties useful as modulator are currently available, see, e.g., Patent Cooperation Treaty (“PCT”) International Publication No. WO 96/11953, entitled “N-Terminally Chemically Modified Protein Compositions and Methods,” herein incorporated by reference in its entirety. This PCT publication discloses, among other things, the selective attachment of water-soluble polymers to the N-terminus of proteins.

A preferred polymer modulator is polyethylene glycol (PEG). The PEG group may be of any convenient molecular weight and may be linear or branched. The average molecular weight of the PEG will preferably range from about 2 kiloDalton (“kD”) to about 100 kDa, more preferably from about 5 kDa to about 50 kDa, most preferably from about 5kDa to about 10 kDa. The PEG groups will generally be attached to the compounds of the invention via acylation or reductive alkylation through a reactive group on the PEG moiety (e.g., an aldehyde, amino, thiol, or ester group) to a reactive group on the inventive compound (e.g., an aldehyde, amino, or ester group).

A useful strategy for the PEGylation of protein consists of combining, through forming a conjugate linkage in solution, a protein and a PEG moiety, each bearing a special functionality that is mutually reactive toward the other. The protein can be prepared with conventional recombinant expression techniques. The proteins are “preactivated” with an appropriate functional group at a specific site. The precursors are purified and fully characterized prior to reacting with the PEG moiety. Ligation of the protein with PEG usually takes place in aqueous phase and can be easily monitored by reverse phase analytical HPLC. The PEGylated protein can be easily purified by preparative HPLC and characterized by analytical HPLC, amino acid analysis and laser desorption mass spectrometry.

Polysaccharide polymers are another type of water soluble polymer which may be used for protein modification. Dextrans are polysaccharide polymers comprised of individual subunits of glucose predominantly linked by α1-6 linkages. The dextran itself is available in many molecular weight ranges, and is readily available in molecular weights from about 1 kD to about 70 kD. Dextran is a suitable water soluble polymer for use in the present invention as a modulator by itself or in combination with another modulator (e.g., Fc). See, for example, WO 96/11953 and WO 96/05309. The use of dextran conjugated to therapeutic or diagnostic immunoglobulins has been reported; see, for example, European Patent Publication No. 0 315 456, which is hereby incorporated by reference. Dextran of about 1 kD to about 20 kD is preferred when dextran is used as a vehicle in accordance with the present invention.

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N— linked and O— linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art. Amino acids that are suitable for glycosylation can be incorporated at specific sites both in the modulator and the protein part. Preferable techniques to use for engineering these specific amino acids are site-directed mutagenesis or comparable method. Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W.H. Freeman & Co., San Francisco), pp. 79-86 (1983). Thus, glycosylation sites in the amyloid α peptide degrading component can be engineered. For example, residues preferably on the surface of neprilysin structure are modified to allow the glycosylation. The 3D structure of neprilysin can be used to select suitable amino acid replacement for the introduction of both glycosylation and pegylation sites. Glycosylation sites are introduced using for example the Asn-X-Ser/Thr sequence. For pegylation, suitable surface exposed amino acids are for example replaced to cystine residues for specific and efficient coupling of the pegylation component.

Compounds of the present invention may be changed at the DNA level, as well. The DNA sequence of any portion of the compound may be changed to codons more compatible with the chosen host cell. For E. coli, which is the preferred host cell, optimized codons are known in the art. Codons may be substituted to eliminate restriction sites or to include silent restriction sites, which may aid in processing of the DNA in the selected host cell. The vehicle, linker and peptide DNA sequences may be modified to include any of the foregoing sequence changes.

Linkers: Any “linker” group is optional. When present, its chemical structure is not critical, since it serves primarily as a spacer. The linker is preferably made up of amino acids linked together by peptide bonds. Thus, in preferred embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines.

The quantitative specificity of proteases varies over a wide range. There are very unspecific proteases known, such as papain which cleaves all polypeptides that contain a phenylalanine, a valine or an leucine residue, or trypsin which cleaves all polypeptides that contain an arginine or a lysine residue. On the other hand, there are highly specific proteases known, such as the tissue-type plasminogen activator (t-PA) which cleaves plasminogen only at a single specific sequence. Proteases with high substrate specificity play an important role in the regulation of protein functions in living organisms. The specific cleavage of polypeptide substrates, for example, activates precursor proteins or deactivates active proteins or enzymes, thereby regulating their functions. Several proteases with high substrate specificities are used in medical applications. Pharmaceutical examples for activation or deactivation by cleavage of specific polypeptide substrates are the application of t-PA in acute cardiac infarction, which activates plasminogen to resolve fibrin clots, or the application of Ancrod in stroke which deactivates fibrinogen, thereby decreasing blood viscosity and enhancing its transport capacity. While t-PA is a human protease with an activity necessary in human blood regulation, Ancrod is a non-human protease. It was isolated from the viper Agkistrodon rhodostoma, and comprises the main ingredient of the snake's poison. Therefore, there exist a few non-human proteases with therapeutic applicability. Their identification, however, is usually highly incidental. The treatment of diseases by administering drugs is typically based on a molecular mechanism initiated by the drug that activates or inactivates a specific protein function in the patient's body, be it an endogenous protein or a protein of an infecting microbe or virus. While the action of chemical drugs on these targets is still difficult to understand or to predict, protein drugs are able to specifically recognize these target proteins among millions of other proteins. Prominent examples of proteins that have the intrinsic possibility to recognize other proteins are antibodies, receptors, and proteases. Although there are a huge number of potential target proteins, only very few proteases are available today to address these target proteins. Due to their proteolytic activity, proteases are particularly suited for the inactivation of protein or peptide targets. When considering human proteins only, the number of potential target proteins is yet enormous. It is estimated that the human genome comprises between 30,000 and 100,000 genes, each of which encodes a different protein. Many of these proteins or peptides are involved in human diseases and are therefore potential pharmaceutical targets. It might be unlikely to find such a protease with a particular qualitative specificity by screening natural isolates. Therefore there is a need to optimize the catalytic selectivity of a known protease or other scaffold proteins including catalytic antibodies.

Selection systems for proteases of known specificity are known in the art, for instance, from Smith et al., Proc. Natl. Acad. Sci. USA, Vol. 88 (1991). As exemplified, the system comprises the yeast transcription factor GAL4 as the selectable marker, a defined and cleavable target sequence inserted into GAL4 in conjunction with the TEV protease. The cleavage separates the DNA binding domain from the transcription activation domain and therewith renders the transcription factor inactive. The phenotypical inability of the resulting cells to metabolize galactose can be detected by a calorimetric assay or by the selection on the suicide substrate 2-deoxygalactose.

Further, selection may be performed by the use of peptide substrates with modifications as, for example, fluorogenic moieties based on groups as ACC, previously described by Harris et al. (US 2002/022243).

Identical or similar approaches could be used in order to identify or produce an effective amyloid β peptide-degrading component as described in this invention. That starting point for the engineering of this amyloid β peptide-degrading component could be an enzyme that possesses some avivity against amyloid β peptide or that have no activity at all. Other components could be a scaffold protein where specific regions are randomized to possess activity against the amyloid β peptide. There are described various scaffold proteins in the literature where one part of the scaffold structure is the core structure holding the randomized part in a relative fixed positions to generate a binding or active site.

Laboratory techniques to generate proteolytic enzymes with altered sequence specificities are in principle known. They can be classified by their expression and selection systems. Genetic selection means to produce a protease or any other protein within an organism which protease or any other protein is able to cleave a precursor protein which in turn results in an alteration of the growth behavior of the producing organism. From a population of organisms with different proteases those having an altered growth behavior can be selected. This principle was reported by Davis et al. (U.S. Pat. No. 5,258,289). The production of a phage system is dependent on the cleavage of a phage protein, which is activated in the presence of a proteolytic enzyme, or antibody which is able to cleave the phage protein. Selected proteolytic enzymes, scaffolds or antibodies would have the ability to cleave an amino acid sequence for activation of phage production.

A system to generate proteolytic enzymes with altered sequence specificities with membrane-bound proteases is reported. Iverson et al. (WO 98/49286) describe an expression system for a membrane-bound protease which is displayed on the surface of cells. An essential element of the experimental design is that the catalytic reaction has to be performed at the cell surface, i.e., the substrates and products must remain associated with the bacterium expressing the enzyme at the surface. Another example of a selection system is the use of FACS sorting (Varadarajan et al., Proc. Natl. Acad. Sci. USA, Vol. 102, 6855 (2005)) that express the active protein on a cell surface and sort cells that contains variants with improved properties. They showed a three million-fold change in specificity for a protease cleavage site.

A system to generate proteolytic enzymes with altered sequence specificities with self-secreting proteases is also known. Duff et al. (WO 98/11237) describe an expression system for a self-secreting protease. An essential element of the experimental design is that the catalytic reaction acts on the protease itself by an autoproteolytic processing of the membrane-bound precursor molecule to release the matured protease from the cellular membrane into the extracellular environment.

Broad et al. (WO 99/11801) disclose a heterologous cell system suitable for the alteration of the specificity of proteases. The system comprises a transcription factor precursor wherein the transcription factor is linked to a membrane-anchoring domain via a protease cleavage site. The cleavage at the protease cleavage site by a protease releases the transcription factor, which in turn initiates the expression of a target gene being under the control of the respective promotor. The experimental design of alteration of the specificity consists in the insertion of protease cleavage sites with modified sequences and the subjection of the protease to mutagenesis.

According to the invention, any protein or peptide can be used directly or as a starting point to generate a suitable amyloid β peptide-degrading component. For example, according to the invention, any protease can be used as first protease. Preferably, any protein or peptide that are of human origin is used, If a natural protein or peptide, normally existing in the human body, is used, the smallest possible changes are preferred. In some methods, two or more fusion proteins with different binding specificities and/or degradation activity are administered simultaneously, in which case the dosage of each fusion protein administered falls within the ranges indicated. Fusion protein is usually administered on multiple occasions. Intervals between single dosages can be, for example, weekly, monthly, every three months or yearly. Intervals can also be irregular as indicated by measuring blood levels of fusion protein in the plasma of the patient. In some methods, dosage is adjusted to achieve a plasma fusion protein concentration of 1-1000 ug/ml and in some methods 25-300 ug/ml. Also in some methods, dosage is adjusted to achieve a plasma fusion protein concentration of 1-1000 ng/ml and in some methods 25-300 ng/ml. Alternatively, fusion protein can be administered as a sustained release formulation, in which case less frequent administration is required. Dosage and frequency vary depending on the half-life of the fusion protein in the patient. In general, fusion protein with an Fc part shows a long half-life. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime. It is predicted that a catalytic active amyloid p peptide degrading fusion protein can be administrated at a lower dose compare to a binding agent such as for example and antibody.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

One aspect of the present invention is the possibility to modify natural wild type proteins to become even more selective in the degradation of amyloid β peptide. Site-directed mutagenesis can be used to introduce/replace amino acids in the wild type sequence. On approach is to use rational design by investigating the active site of the degrading enzymes. Amino acids that potentially will alter the selectivity profile (degradation of amyloid β peptide compare to other peptides/proteins) can be replace with other amino acids a the new variants can be tested in cleavage assays known in the art. Preferably, variants that have a higher catalytic degradation activity towards amyloid β peptide compare to other related peptides are useful. Other related peptides include but are not limited to Enkephalin, Neuropeptide Y, Substance P, somastatin, cholecystokinin.

It is an object of the present invention to provide methods and materials, which are suited for the development of a treatment for neurodegenerative diseases and for the identification of compounds useful for therapeutic intervention in such diseases. Based on the finding that β-amyloid can be clearance through an optimized enzymatic-mediated mechanism the present invention sets out for providing such methods and materials as laid out in the claims section and described hereinafter.

The invention provides a method for preventing and treating neurodegenerative disorders comprising administering to the peripheral system of a mammalian an effective amount of an optimized enzymatic active compound. In particular, the enzymatic active compound is a fusion protein where one part has enzymatic activity and the other part regulate the half-life in plasma. The method is suited for preventing and treating brain amyloidosis such as Alzheimer's disease. The invention also provides different assay principles-biochemical and in particular cellular assays for testing an optimized enzymatic compound, preferably screening a plurality of compounds, for modulating activity and plasma half-life.

In a further embodiment, the assay comprises the addition of a known inhibitor of the member of the neprilysin family before detecting said enzymatic activity. Suitable inhibitors are e.g. phosphoramidon, thiorphan, spinorphin, or a functional derivative of the foregoing substances.

In a general sense, assays according to the invention measure the enzymatic activity and half-life in plasma, both in vitro and in vivo.

In another aspect, the present invention provides a method for producing a medicament comprising the steps of (i) identifying a compound which degrades Aβ-peptides, preferably a compound that is highly specific and with high Aβ-peptides degrading activity (ii) linking this Aβ-peptides degrading compound to a modulator compound that determine the half-time in plasma.

The compounds of this invention may be made in transformed host cells using recombinant DNA techniques. To do so, a recombinant DNA molecule coding for the fusion protein is prepared. Methods of preparing such DNA molecules are well known in the art. For instance, sequences coding for the modulator and protein could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the modulator, protein or fusion in an appropriate host. The vector comprises the DNA molecule that codes for the modulator, protein and/or fusion operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector having the DNA molecule thereon is used to transform an appropriate host. This transformation may be performed using methods well known in the art. Any of a large number of available and well-known host cells may be used in the practice of this invention. The selection of a particular host is dependent upon a number of factors recognized by the art. These include, for example, compatibility with the chosen expression vector, toxicity of the fusion encoded by the DNA molecule, rate of transformation, ease of recovery of the fusion, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the fusion is purified from culture by methods well known in the art. One preferably approach is to use Protein A or similar technique to purify the fusion protein when using a Fc part as a modulator. The modulator, protein and fusion may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides or proteins since it is the most cost-effective method of making small peptides or proteins.

In general, the compounds of this invention have pharmacologic activity resulting from their ability to degrade the amyloid β peptide in vivo. The activity of these compounds can be measured by assays known in the art. For the NEP-Fc compounds, in vivo assays are further described in the Examples section herein.

In general, the present invention also provides the possibility of using pharmaceutical compositions of the inventive compounds. Such pharmaceutical compositions may be for administration for injection, or for oral, pulmonary, nasal, transdermal or other forms of administration. In general, the invention encompasses pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HC1, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations. These administration alternatives are well known in the art.

The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.1-1000 micrograms of the inventive compound per kilogram of body weight, preferably 0.1-150 micrograms per kilogram.

### EXAMPLES

### Example 1

### Fusion Protein Construction

### Description of the Protein Domains

The extra cellular domain of Neprilysin is defined as amino acid 51-749 (excluding the first Methionine) (SEQ ID No 1-4). There are two polymorphisms that lead to amino acid difference identified in this domain, and the amino acid sequence for the different variants are described in SEQ ID no 1-4. The extracellular domain of Neprilysin is fused to the IgG4 Fc domain (including the hinge region). A signal sequence (SEQ ID 5) is introduced to enable secretion of the protein into the culture media during expression. The sequence of the hinge region is shown in SEQ ID 6 and the IgG4 Fc domain is shown in SEQ ID 7. The complete fusion protein (with a human Neprilysin variant corresponding to SEQ ID 1) is described in SEQ ID 8. The final fusion protein (excluding the signal sequence) has a predicted molecular weight of 211 kDa (as a dimer).

### Description of the Construction of the Gene Encoding the Fusion Protein

The gene encoding the extra cellular domain of Neprilysin as fusion to the gene encoding Fc domain of IgG4, is introduced into a pGEM cloning vector. The molecular biology work is PCR based, using primers specific for the 3′ and the 5′ end of the genes included. Forward primer for amplifying human Neprilysin extra cellular domain is shown in SEQ ID 9, where the first GTA sequence is added to create a BstZ17I unique blunt-end restriction site (GTATAC) that will be use for cloning the signal peptide. The reverse primer used (SEQ ID 10) contains a sequence that corresponds to the sequence of the hinge of IgG4 (SEQ ID 11). This is to create an overlap region with IgG4.

The human IgG4 Fc domain is amplified using PCR with forward primer as shown in SEQ ID 12. This primer contains a part that corresponds to the C-terminus part of human Neprilysin (SEQ ID 13), to create an overlapping region with Neprilysin. Reverse primer for amplifying IgG4 Fc domain (SEQ ID 14) contains a sequence that corresponds to the ATTB2 sequence for Gateway cloning and a stop codon (SEQ ID 15).

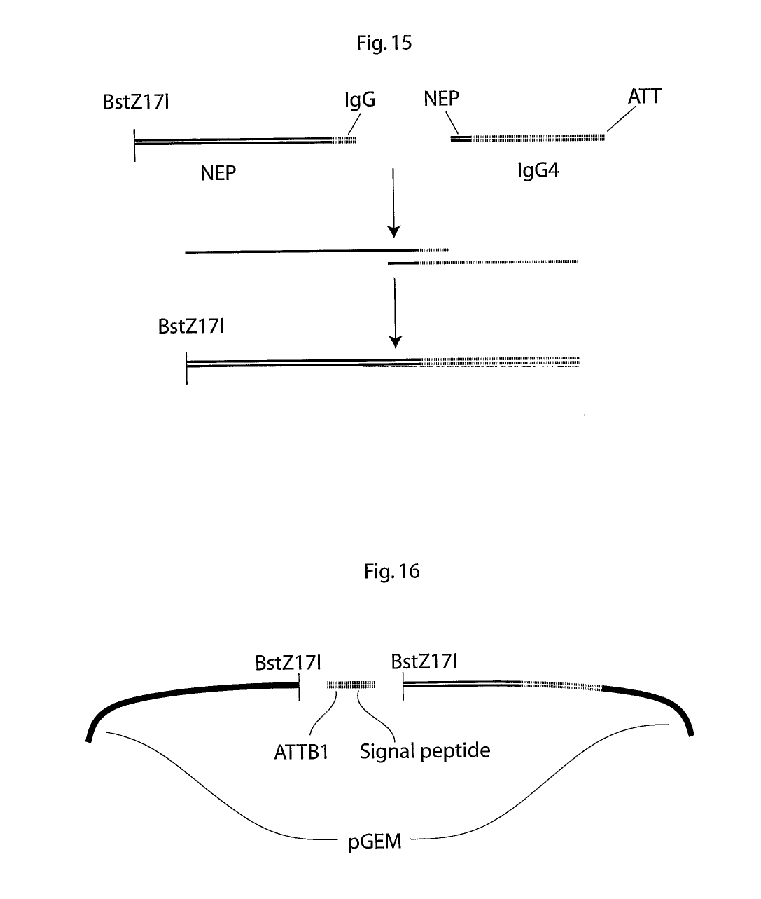

An overlapping PCR reaction using the amplified genes encoding human Neprilysin and Fc IgG4 domain as templates and the primers SEQ ID 9, 10, 12, and 14 (the primers SEQ ID 10 and 12 has an overlapping specificity) created a fragment corresponding to the gene encoding the Neprilysin-Fc fusion protein (an overview is shown in FIG. 15). Addition of the murine kappa light chain signal peptide is performed by ligating a synthetic DNA oligo into the pGEM cloning vector upstream of the gene encoding the Neprilysin-Fc fusion protein using the BstZ17I blunt-end restriction site. The sequence of the signal peptide is amplified using a forward primer (SEQ ID 16) that contains a sequence (SEQ ID 17) that corresponds to the ATTB1 Gateway consensus sequence followed by an enhancer sequence, ribosome binding site, a TATA box, Kozak sequence and a start codon. The reverse primer used is shown in SEQ ID 18. The strategy for introducing the signal sequence is shown in FIG. 16.

The complete gene (encoding the Nep-Fc fusion protein and the signal sequence) is initially inserted into a pGEM-vector, and subsequently into a Gateway donor vector. The Gateway donor vector is used to introduce the complete Nep-Fc gene into several expression vectors. By using the Gateway system, the transfer from donor vector to the expression vectors can be done in an efficient way by using recombination instead of restriction enzymes. The mammalian expression vectors investigated are primarily pCEP4, pEAK10 and pcDNA3.1 (Gateway adapted). All these are standard mammalian expression vectors based on a CMV promoter. The genes are sequenced after all cloning steps to verify the DNA sequence.

### Example 2

### Expression and Purification the Fc-Neprilysin Protein

The Nep-Fc fusion protein is transiently expressed in mammalian cells. Several cell lines are used, including HEK293T and HEK293EBNA cells. The expression of the fusion protein is performed in suspension-adapted cells or adherent cell cultures and is investigated using different transfection reagents, different cell densities and different ratio of transfection reagents and plasmid. The activity of the fusion protein is verified in small-scale optimisation experiments. The Nep-Fc is purified directly from the culture supernatants using Protein A affinity chromatography as a primary step. When a second purification step is needed, e.g. ion exchange or gel filtration is used. The final fusion protein is formulated in a buffer suitable for in vivo use (mouse studies) e.g. a buffer including a stabilising agent (e.g. sucrose, salt or detergent). The final purified fusion protein is analysed for concentration (e.g. A280, BCA), identity (e.g. western blot using Neprilysin or IgG4 specific antibodies, Mass spectrometry) and purity (e.g. SDS-PAGE, Analytical gel filtration). To ensure processing of the signal peptide, the protein is analysed by N-terminal sequencing. The final protein batch is used in in vitro and in vivo studies to verify function.

### Example 3

### NEP-FC Enzyme Activity

The recombinant NEP-Fc was evaluated for neprilysin enzymatic activity using a two-step chromogenic assay. In the first reaction glutaryl-Ala-Ala-Phe-4-methoxy-2-naphthylamide is cleaved by neprilysin to Phe-4-methoxy-2-naphthylamide, while in the second step an aminopeptidase is used to generate the fluorescent 4-methoxy-2-naphthylamine. Reaction mixtures in 100 μL volumes containing 100 μM glutaryl-Ala-Ala-Phe-4-methoxynaphthylamide, 50-100 μg membrane fraction, and 20 mM MES buffer were added to a 96 well microtiterplate. Incubations were for 2 hours at 37° C. in a water bath. At the end of the incubation period, the reaction was terminated by the addition of phosphoramidon. Leucine aminopeptidase was added and the mixtures were incubated for an additional 15 minutes. The 4-methoxy-2-naphthylamine was quantified spectrofluorimetrically at an excitation wavelength of 340 nM and an emission wavelength of 425 nM. Free 4-methoxynaphthylamine was used to construct a standard curve.

### Example 4

### Amyloid β Peptide Concentration Measurements

This assay describes the measurement of the amyloid β peptide. The assay is based on two antibodies that detect the amyloid β peptide when its not degraded by a protein or enzyme. This particular example describes the use of supernatant from cells but can be applied on various samples such as pure buffer or plasma.

HEKAPP is harvested when the cells are 80-90% confluence. The cells are Seeded at a conc of 0.2×10⁶/ml in DMEM to a 96-well poly-D-Lysine coated plate (BD, Falcon). Use multidrop, 100 ul cell susp/well. Incubated cell plates o/n at 37° C., 5% CO2. NEP-Fc is incubated for 24 h (or any suitable time) at 37° C., 5% CO2. Then 100 μl of medium is transferred to a round bottom 96-well plate (Greiner, polypropylene). 50 μl detection solution (Need 5 ml of 0.5 μg/ml of the RαAβ40 and 0.25 μg/ml biotin-4G8 in IGEN-assay buffer. RαAβ40: Stock: 1,22 mg/ml RαAβ40 (Biosource 44-348). Add 2 μl stock RαAβ40+5000 μl IGEN assay buffer. Final concentration in assay 0.125 μg/ml) Biotin-4G8: Stock: 1 mg/ml bio-4G8 Add 1.25 μl stock Bio-4G8 to RαAβ40 solution from above Final concentration in assay 0.063 μg/mlis added and incubate at 4° C.>7 h). Then add 50 μl Dynabead & Ru-GαR solution and shake (vigorously) at 22° C. for 1 h. Finally, read the plate in the IGEN/Bio Veris M8 analyzer program Aβ-cell assay. An example of amyloid β peptide concentration measurement was carried out where the standard curve experiment has a good correlation (R²=0.9959).

### Example 5

### Catalytic Degradation of Amyloid β Peptide Change the Equilibrium Between the Free Pools of Amyloid β Peptide

An experiment is performed to investigate the possibility to remove amyloid β peptide from one compartment by degradation in another compartment connected through a membrane. A similar experiment was conducted using an antibody as an amyloid β peptide agent. The experiment is described by DeMattos et al., (PNAS, 2001). By degrading the amyloid β peptide with neprilysin and/or NEP-Fc in one compartment the concentration of amyloid β peptide in the other compartment is decreased. Importantly, neprilysin or NEP-Fc cannot cross the membrane due to its high molecular weight (161.000 Da and 211.000 Da, respectively).

### Example 6

### Inhibition of Deposition of Aβ₁₋₄₀ Fragments on Amyloid Plaques

A protocol was used where amyloid beta 1-40 (Aβ₁₋₄₀) is initially deposited onto a 96 well microtiter plate. Radioactive (¹²⁵I labeled) Aβ₁₋₄₀ is then added to the wells of this plate where it further adds to the Aβ₁₋₄₀ deposited. This mimics the deposition of Aβ₁₋₄₀ seen in the brains of Alzheimer patients. The object of this experiment was to see if neprilysin could break down Aβ₁₋₄₀ into fragments that are no longer deposited on the amyloid plaques. This demonstrates that neprilysin could prevent the continued formation of amyloid deposits in Alzheimer's disease.

A 96 well plates is pre-coated with Aβ₁₋₄₀. In the control, 100 pM of ¹²⁵I-Aβ₁₋₄₀ was deposited onto the pre-deposited Aβ₁₋₄₀ plaque for three hours. Neprilysin is added at concentrations of 500 ng, 50 ng and 5 ng to the wells along with ¹²⁵I-Aβ₁₋₄₀ for three hours. Inhibition of deposition of ¹²⁵I-Aβ₁₋₄₀ is then detected for the various concentration of neprilysin.

### Example 7

### Determination of Sites of Cleavage of Aβ Peptides

Purified neprilysin and/or NEP-Fc is incubated with 25 μM Aβ₁₋₄₀ in 40 mM potassium phosphate buffer, pH 7.2, at 37° C. for 1 hour. The reaction products are loaded onto a C₄ reverse phase HPLC column and products resolved using a linear gradient of 5 to 75% acetonitrile over 65 minutes. Products are detected by absorbance at 214 nm using a Waters 484 detector and individual product peaks is collected manually. Product analysis can also be conducted on an intact reaction mixture in which products were not resolved by HPLC. Products are identified by matrix assisted laser desorption ionization time of flight mass spectrometry (MALDI-TOF-MS). The reaction of neprilysin and/or NEP-Fc with Aβ₁₋₄₂ are conducted in a similar manner with products identified by MALDI-TOF-MS directly from reaction mixtures.

### Example 8

### Aβ₁₋₄₀ Deposition Assay

Beta amyloid deposition assays are conducted as described by Esler et. al. (Esler et al. (1997) Nat Biotech 15:268-263). Briefly, 96 well microtiter plates pre-coated with aggregated amyloid β₁₋₄₀ (QCB/Biosource, Hopkinton, Mass.) are additionally coated with 200 μl of a 0.1% bovine serum albumin solution in 50 mM Tris-HCl, pH 7.5 for 20 minutes to prevent non-specific binding. For measuring Aβ₁₋₄₀ deposition in the presence or absence of neprilysin, a 150-μl solution of 0.1 nM ¹²⁵I labeled Aβ₁₋₄₀ in 50 mM Tris-HCl, pH 7.5 is added to the pre-coated well and incubated for four hours. When added, neprilysin (0.5 to 500 ng) is placed directly in the well at zero time. The reaction is stopped by washing off excess undeposited radiolabeled Aβ₁₋₄₀ with 50 mM Tris-HCl, pH 7.5. The radiolabel deposited onto the washed well is counted in a gamma counter. In a variation of this protocol, neprilysin can be preincubated with 1 nM ¹²⁵I-Aβ₁₋₄₀ for 60 minutes and then added to the deposition assay.

### Example 9

### Neuoroprotection Assays

Neurotoxicity assays are performed as described by Estus et. al. (Estus et al. (1997) J Neurosci 17:7736-7745) using embryonic day 18 rat fetuses to establish primary rat cortical neuron cultures. Rat brain cortical cells are initially cultured in AM₀ media for 3-5 hrs in 16 well chamber slides (Nalge Nunc International, Rochester, N.Y.) pre-coated with polyethyleneimine at a density of 1×10⁵ cells per well. The culture is enriched in neurons by replacement of the AM₀ media with Dulbecco's modified Eagle's medium (DMEM, Life Technologies, Rockville, Md.) containing 100 units/ml penicillin, 100 μg/ml streptomycin and 2% B27 serum supplement (Life Technologies, Rockville, Md.). Cells are treated with Aβ peptides and then fixed with 4% paraformaldehyde for 15 min. at room temperature.

After washing the cells with PBS they are then stained with Hoechst 33258 at 1 μg/ml for 10 minutes. Neurons are then visualized by fluorescence microscopy. Those cells with uniformly dispersed chromatin are scored as survivors, while those cells containing condensed chromatin are scored as non-survivors. Readings are typically taken in triplicate with a minimum of 250 neurons scored from each well. Cells treated as described above are visualized using a Nikon microscope equipped with a Hoffman modulation contrast lens. Statistical analysis is performed on the samples using ANOVA. The same treatment with the Aβ peptides is performed but the Aβ peptides have been pretreated with neprilysin or NEP-Fc. This shows that the degradation process removes the toxic effect of the Aβ peptides.

### Example 10

### Description of the Protein Domain IDE, IDE Fc (IgG4), ECE1 and ECE1 Fc(IgG4)

IDE (insulin degrading enzyme) is a 1018 amino acid long protein (SEQ ID NO 19). There are splice variants and polymorphism variants described of IDE. In one splice variant, one exon is replaced with another exon of the same size, encoding a peptide sequence similar to the “wt” exon (described in SEQ ID NO 20). This variant has been described to be less efficient in degrading both insulin and Aβ. There are also several polymorphisms in the IDE gene described, that lead to amino acid difference identified in this domain: D947N (SEQ ID NO 21), E612K (SEQ ID NO 22), L298F (SEQ ID NO 23) and E408G (SEQ ID NO 24).

The extra-cellular domain of ECE1 (endothelin-converting enzyme 1) (SEQ ID 26) is a 681 amino acids long protein, defined as amino acid 90-770 of the full-length, membrane-bound ECE1 protein.

During construction of the final fusion protein, IDE and ECE1 (extra-cellular domain) are fused to the IgG4 Fc domain (including the hinge region), with a signal sequence (SEQ ID NO 5) added in the N-terminal end of the fusion protein. The signal sequence will enable secretion of the protein into the culture media during expression. The sequence of the

### IDE Protein, Variant 1

MRYRLAWLLHPALPSTFRSVLGARLPPPERLCGFQKKTYSKMNNPAIKRI
GNHITKSPEDKREYRGLELANGIKVLLMSDPTTDKSSAALDVHIGSLSDP
PNIAGLSHFCEHMLFLGTKKYPKENEYSQFLSEHAGSSNAFTSGEHTNYY
FDVSHEHLEGALDRFAQFFLCPLFDESCKDREVNAVDSEHEKNVMNDAWR
LFQLEKATGNPKHPFSKFGTGNKYTLETRPNQEGIDVRQELLKFHSAYYS
SNLMAVCVLGRESLDDLTNLVVKLFSEVENKNVPLPEFPEHPFQEEHLKQ
LYKIVPIKDIRNLYVTFPIPDLQKYYKSNPGHYLGHLIGHEGPGSLLSEL
KSKGWVNTLVGGQKEGARGFMFFIINVDLTEEGLLHVEDIILHMFQYIQK
LRAEGPQEWVFQECKDLNAVAFRFKDKERPRGYTSKIAGILHYYPLEEVL
TAEYLLEEFRPDLIEMVLDKLRPENVRVAIVSKSFEGKTDRTEEWYGTQY
KQEAIPDEVIKKWQNADLNGKFKLPTKNEFIPTNFEILPLEKEATPYPAL
IKDTVMSKLWFKQDDKKKKPKACLNFEFFSPFAYVDPLHCNMAYLYLELL
KDSLNEYAYAAELAGLSYDLQNTIYGMYLSVKGYNDKQPILLKKIIEKMA
TFEIDEKRFEIIKEAYMRSLNNFRAEQPHQHAMYYLRLLMTEVAWTKDEL
KEALDDVTLPRLKAFIPQLLSRLHIEALLHGNITKQAALGIMQMVEDTLI
EHAHTKPLLPSQLVRYREVQLPDRGWFVYQQRNEVHNNCGIEIYYQTDMQ
STSENMFLELFCQIISEPCFNTLRTKEQLGYIVFSGPRRANGIQSLRFII
QSEKPPHYLESRVEAFLITMEKSIEDMTEEAFQKHIQALAIRRLDKPKKL
SAECAKYWGEIISQQYNFDRDNTEVAYLKTLTKEDIIKFYKEMLAVDAPR
RHKVSVHVLAREMDSCPVVGEFPCQNDINLSQAPALPQPEVIQNMTEFKR
GLPLFPLVKPHINFMAAKL

### SEQ ID NO 20

### IDE Protein, Variant 2 (Splice Variant)

MRYRLAWLLHPALPSTFRSVLGARLPPPERLCGFQKKTYSKMNNPAIKRI
GNHITKSPEDKREYRGLELANGIKVLLMSDPTTDKSSAALDVHIGSLSDP
PNIAGLSHFCEHMLFLGTKKYPKENEYSQFLSEHAGSSNAFTSGEHTNYY
FDVSHEHLEGALDRFAQFFLCPLFDESCKDREVNAVDSEHEKNVMNDAWR
LFQLEKATGNPKHPFSKFGTGNKYTLETRPNQEGIDVRQELLKFHSAYYS
SNLMAVCVLGRESLDDLTNLVVKLFSEVENKNVPLPEFPEHPFQEEHLKQ
LYKIVPIKDIRNLYVTFPIPDLQKYYKSNPGHYLGHLIGHEGPGSLLSEL
KSKGWVNTLVGGQKEGARGFMFFIINVDLTEEGLLHVEDIILHMFQYIQK
LRAEGPQEWVFQECKDLNAVAFRFKDKERPRGYTSKIAGILHYYPLEEVL
TAEYLLEEFRPDLIEMVLDKLRPENVRVAIVSKSFEGKTDRTEEWYGTQY
KQEAIPDEVIKKWQNADLNGKFKLPTKNEFIPTNFEILPLEKEATPYPAL
IKDTVMSKLWFKQDDKKKKPKACLNFEFFSPFAYVDPLHCNMAYLYLELL
KDSLNEYAYAAELAGLSYDLQNTIYGMYLSVKGYNDKQPILLKKIIEKMA
TFEIDEKRFEIIKEAYMRSLNNFRAEQPHQHAMYYLRLLMTEVAWTKDEL
KEALDDVTLPRLKAFIPQLLSRLHIEALLHGNITKQAALGIMQMVEDTLI
EHAHTKPLLPSQLVRYREVQLPDRGWFVYQQRNEVHNNCGIEIYYQTDMQ
STSENMFLELFCQIISEPCFNTLRTKEQLGYIVFSGPRRANGIQSLRFII
QSEKPPHYLESRVEAFLITMEKSIEDMTEEAFQKHIQALAIRRLDKPKKL
SAECAKYWGEIISQQYNFDRDNTEVAYLKTLTKEDIIKFYKEMLAVDAPR
RHKVSVHVLAREMDSCPVVGEFPCQNDINLSQAPALPQPEVIQNMTEFKR
GLPLFPLVKPHINFMAAKL

hinge region is shown in SEQ ID NO 6 and the IgG4 Fc domain is shown in SEQ ID NO 7. The complete IDE-Fc(IgG4) fusion protein (with an human IDE variant corresponding to SEQ ID NO 19) is described in SEQ ID NO 25. The final fusion protein IDE-Fc (IgG4) has a predicted molecular weight of 147 kDa (as a monomer) or 294 kDa as a dimer. ECE1-Fc (IgG4) fusion protein (with an human ECE1 variant corresponding to SEQ ID NO 26) is described in SEQ ID NO 27 and has a predicted molecular weight of 103 kDa (as a monomer) or 206 kDa (as a dimer).

### Description of the Protein Domain Neprilysin-Fc (IgG2)

In a similar fashion as described in Example 1, the extra cellular domain of Neprilysin (SEQ ID NO 1) is fused to the IgG2 Fc domain (including the hinge region). A signal sequence is added (SEQ ID NO 5) in the N-terminal end of the fusion protein, to enable secretion of the protein into the culture media during expression. The sequence of the IgG2 hinge region is shown in SEQ ID NO 28 and the IgG2 Fc domain is shown in SEQ ID NO 29. The complete Neprilysin-Fc (IgG2) fusion protein (with an human Neprilysin variant corresponding to SEQ ID NO 1) is described in SEQ ID NO 30. The final fusion protein Neprilysin-Fc (IgG2) has a predicted molecular weight of 105.5 kDa (as a monomer) or 211 kDa as a dimer.

### Description of the Construction of the Gene Encoding the Fusion Protein IDE-Fc (IgG4)

The gene encoding human IDE is PCR amplified (oligonucleotides SEQ ID NO 31 and SEQ ID NO 32) from human skeletal muscle cDNA (Clontech cat# 637234) and cloned in a pGEM-T cloning vector. A human kappa light chain signal sequence is then introduced at the 5′ end of IDE using PCR (oligonucleotides SEQ ID NO 33 and SEQ ID NO 34) and the product is cloned in a pGEM-T vector. The human IgG4 Fc domain is similarly PCR amplified (oligonucleotides SEQ ID NO 35 and SEQ ID NO 36) from an in-house plasmid (Nep-IgG4, described in Example 1) and cloned in pGEM-T. The last codon of the IDE gene and the first codon of the IgG4 hinge region form a unique XhoI site. This site is utilized to transfer IgG4 to the pGEM-T-IDE plasmid and generate a fusion construct. A secondary PCR is performed to add attB sites at both ends of the fusion gene (oligonucleotides SEQ ID NO 37 and SEQ ID NO 38). The PCR product is introduced into pDONR221 using Gateway BP recombination. The resulting entry clone is sequence verified and used to transfer the fusion gene using Gateway LR recombination to the pCEP4/GW and pEAK10/GW expression vectors.

### Description of the Construction of the Gene Encoding the Fusion Protein ECE1-Fc (IgG4)

The gene encoding human ECE1 is PCR amplified from an in-house cDNA source (plasmid DNA) using oligonucleotides that introduce a human kappa light chain signal sequence at the 5′ end and a unique EcoRI site at the 3′ end (oligonucleotides SEQ ID NO 39 and SEQ ID NO 40). The human IgG4 Fc domain is similarly PCR amplified from plasmid DNA but with a EcoRI site at the 5′ end (oligonucleotides SEQ ID NO 41 and SEQ ID NO 36). ECE1 and IgG4 are separately cloned in a pGEM-T vector. The EcoRI site is utilized to transfer IgG4 to the pGEM-T-ECE1 plasmid and generate a fusion construct. Site-directed mutagenesis (oligonucleotides SEQ ID NO 42 and SEQ ID NO 43) is then used to regenerate the original sequence and delete the EcoRI site. PCR is performed to add attB recombination sites at both ends of the fusion gene (oligonucleotides SEQ ID NO 37 and SEQ ID NO 38). The PCR product will be introduced into pDONR221 using Gateway BP recombination. The resulting entry clone will be sequence verified and used to transfer the fusion gene to the pCEP4/GW and pEAK10/GW expression vectors.

### Description of the Construction of the Gene Encoding the Fusion Protein Nep-Fc (IgG2)

The gene encoding the fusion protein NEP-Fc (IgG2) was generated by merging 2 overlapping PCR fragments. The first fragment corresponds to the soluble domain of NEP and was amplified from an in-house plasmid (pGEM-NEP-IgG4, described in example 1) using a forward primer that corresponds to the Gateway attB 1 consensus sequence (oligonucleotides SEQ ID NO 44) to include the kappa light chain signal peptide at the 5′ end of NEP and a reverse primer (oligonucleotides SEQ ID NO 45) that includes the end of the coding region of NEP (without the stop codon) following the sequence corresponding to the hinge region of human IgG2 to create an overlapping region with the second fragment. The second fragment corresponds to the hinge region and the Fc domain of human IgG2 and was amplified using oligonucleotides that introduce the last 25 coding nucleotides of NEP at the 5′ end (to create overlapping region with the first fragment) and the attB2 consensus sequence at the 3′ end (SEQ C and D). Oligonucleotides SEQ ID NO 46 and oligonucleotides SEQ ID NO 47 were use for the final overlapping PCR and the fragment was then introduced into pDONR211 using Gateway BP recombination. The resulting entry clone is sequence verified and used to transfer the fusion gene using Gateway LR recombination to the pCEP4/GW and pEAK10/GW expression vectors.

### Description of the Construction of the Gene Encoding Neprilysin

The gene coding for the soluble domain of NEP was amplified from an in-house plasmid (pGEM-NEP-IgG4, described in example 1) with a forward oligonucleotide corresponding to the attB 1 consensus sequence (oligonucleotides SEQ ID NO 48) to include the kappa light chain signal peptide and a reverse primer introducing a stop codon (TGA) and the is attB2 consensus sequence at the 3′ end (oligonucleotides SEQ ID NO 49). The fragment was then introduced into pDONR221 using Gateway BP recombination. The resulting entry clone is sequence verified and used to transfer the fusion gene using Gateway LR recombination to the pCEP4/GW and pEAK10/GW expression vectors.

### Example 11

### Expression of Extra Cellular Domain of Neprilysin and Fusion Proteins Neprilysin-Fc (IgG4), Neprilysin-Fc(IgG2) and IDE-Fc(IgG4)

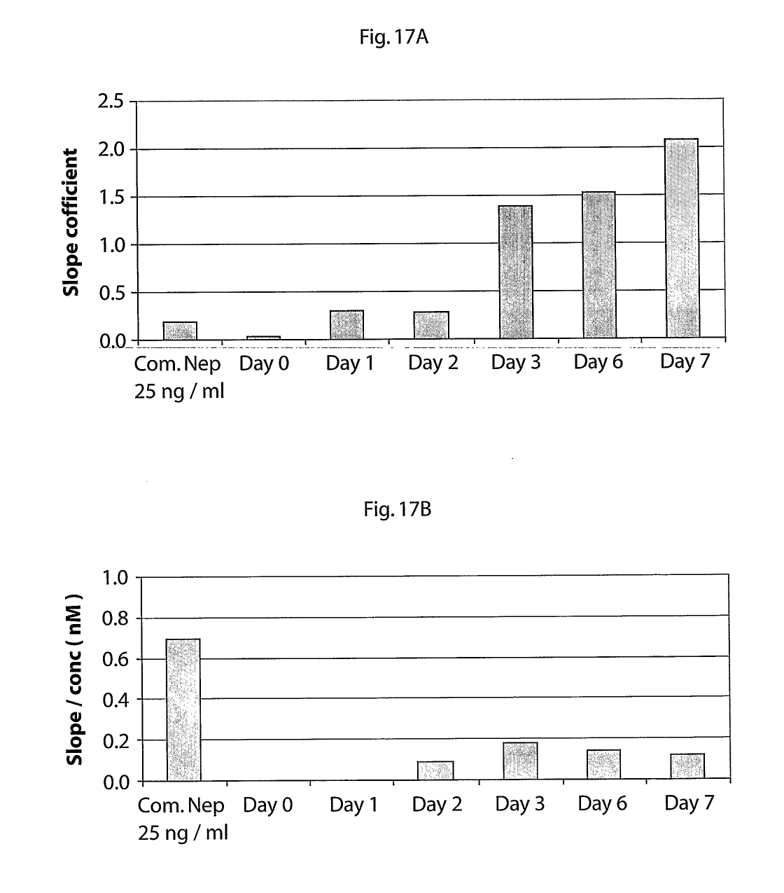

The proteins Neprilysin (extra-cellular domain only), Nep-Fc (IgG4), Nep-Fc (IgG2) and IDE-Fc (IgG4) are transiently expressed in suspension-adapted mammalian cells. The cell lines used in the production experiments are cell lines derived from HEK293, including HEK293S, HEK293S-T and HEK293S-EBNA cells. Expression from plasmids pCEP4 and PEAK 10 encoding the protein of interest is tested. Transfection it performed at cell density of approximately 0.5−1×10⁶ and with plasmid DNA at concentrations ranging from 0.3-0.8 μg/ml cell suspension (final concentration). Transfection reagents that were tested are Polyethylenimine (Polyscience) at 2 μg/ml cell suspension (final concentration) and RO1539 (Roche) at 1 μl/ml cell suspension (final concentration). Expression was performed in cell culture volumes of 200 ml in shaker flasks (for proteins Nep-Fc(IgG4) and IDE-Fc(IgG4)), 400 ml spinner flasks (proteins Neprilysin and Neprilysin-Fc(IgG2)), 1 L Bioreactor (for fusion protein Nep-Fc(IgG4)), 5 L Bioreactor (for Neprilysin protein) and 10 L Bioreactors (for the fusion protein Neprilysin-Fc(IgG4)). Expression was followed by taking samples from the culture supernatants at different days and analyzing cell density, cell viability, protein expression and enzyme activity. Cell cultures were harvested after 7 or 10 days by centrifugation, and the cell culture media was used in protein purification experiments. Results from analyzing activity in culture media from 5 L Bioreactor producing Neprilysin is shown in FIG. 17. All combinations of transfection reagents and plasmid concentrations were successful, giving different level of production, typically in the range of 2-3 mg/L.

### Example 12

### Serum-Free Expression of Neprilysin

The extra cellular domain of Neprilysin was transiently expressed from expression vector pCEP4-Nep under serum-free conditions in suspension-adapted mammalian cells (293-F and HEK293S-EBNA).

Transfection was performed at cell density 0.5−1×10⁶ and with DNA concentrations in the range of 0.3-0.8 μg/ml cell suspension (final concentration). The transfection reagents used are 293 fectin (InVitrogen) at 1.3 μl/ml cell suspension (final concentration), Polyethylenimine (Polyscience) at 2 μg/ml cell suspension (final concentration) and RO1539 (Roche) at 1 μl/ml cell suspension (final concentration). Expression was performed in 200 ml scale (shaker flasks).

Expression and cell growth was followed by taking samples at different days and analyze cell density, cell viability, protein expression and enzyme activity. The cell cultures were harvested after 7 days by centrifugation, and the cell media with expressed protein was used in protein purification experiments.

Expression levels were typically in the range of 1-2 mg/L, except for in the cultures transfected with RO1539, when the expression was below detection levels or very low (<0.5 mg/L).

### Example 13

### Purification of Expressed Neprilysin Protein by Solid-Phase Extraction

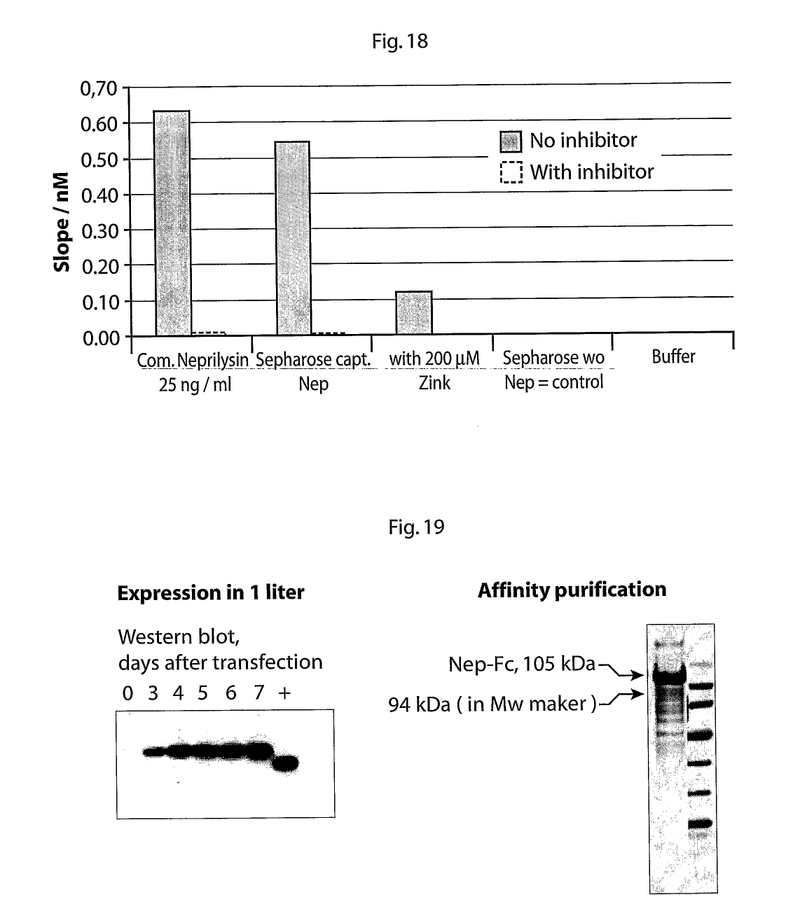

Neprilysin is affinity purified directly from the culture supernatants. Soluble Neprilysin is purified using biotinylated anti-neprilysin antibody (R&D Systems) bound to streptavdin sepharose (GE Healthcare). 14 μg biotinylated anti-neprilysin antibody (R&D systems) was added to 140 μl streptavidin sepharose slurry (GE Healthcare) and incubated during gentle mixing for 2 hours, in room temperature. The sepharose was washed twice and resuspended in 140 μl PBS. The sepharose slurry with bound anti-neprilysin antibody, was added to 10 ml culture supernatant and the sample was incubated for 5 hours at 4° C. After incubation, the sepharose was washed once with PBS and resuspended in 1200 μl 50 mM Tris-HCl, pH 7.5, 150 mM NaCl. 600 μl slurry was used for activity measurement and the other 600 μl slurry was pelleted and resuspended in 60 μl 0.1 M citrate, pH 3.2, and incubated for 10 minutes in room temperature. The sepharose was pelleted and the concentration of purified protein in the supernatant was measured by absorbance at 280 nm. The activity of the purified protein is shown in FIG. 18. The purification of neprilysin was also performed according to description above but with addition of 200 μM ZnCl₂. There was no difference in activity between the sample with and without added zink. The differences in the figure depend on uncertainty in concentration measurements.

### Example 14

### Purification of Expressed IDE-Fc Protein by Solid-Phase Extraction

IDE-Fc is purified using Protein A sepharose (GE Healthcare). 100 μl protein A sepharose was added to 50 ml cell supernatant and incubated at 4° C. over night. After incubation, the sepharose was washed once with PBS and resuspended in 1200 μl 50 mM Tris-HCl, pH 7.5, 150 nM NaCl. 600 μl slurry was used for activity measurement and the other 600 μl slurry was pelleted and resuspended in 60 μl 0.1 M citrate, pH 3.2, and incubated for 10 minutes in room temperature. The sepharose was pelleted and the concentration of purified protein in the supernatant was measured by absorbance at 280 nm.

### Example 15

### Purification of Expressed Fc-Fusion Proteins by Affinity Chromatography and Low pH Elution

Purification of the fusion proteins was performed using cell media from expression in mammalian cells. The purification was performed by Affinity chromatography (Protein A) followed by low pH elution, and was performed on an ÄKTAExplorer Chromatography system (GE Healthcare). rProtein A Sepharose FF (GE Healthcare), approximately 2 ml in a XK16 column (GE Healthcare) was equilibrated with 20 ml PBS (2.7 mM KCl, 138 mM NaCl, 1.5 mM KH2PO4, 8 mM Na2HPO4-7H2O, pH 6.7-7.0, Prepared from 10× stock, Invitrogen). 50 ml cell culture media with expressed fusion proteins (Neprilysin-Fc (IgG2), Neprilysin-Fc(IgG4) or IDE-Fc(IgG4)) was applied on the column at 1 ml/minute. The columns were washed with 50 ml PBS before the bound protein was eluted with. Elution buffer (0.1 M Citric Acid, pH 3.2). Purified fractions were immediately neutralized by adding 200 μl M Tris Base to 1 ml eluted protein. Purified fractions were pooled, and buffer of the pooled protein was exchanged to 50 mM Tris-HCl, pH 7.5, 150 mM NaCl using centrifuge filters (Amicon, Mw cut off 5 kDa). Purified protein was analyzed on SDS-PAGE, and was found to be approximately 90% pure. An example of purified Neprilysin protein fused to a Fc part is show in FIG. 19.

### Example 16

### Purification of Neprilysin-Fc Protein (IgG2 and IgG4) and IDE-Fc (IgG4) Protein Using High Salt Elution

Purification of the expressed fusion proteins was performed using cell media from expression in mammalian cells. The purification was essentially performed as described in Dwyer et al 1999. 1 ml HiTrap Protein A columns (GE Healthcare) were equilibrated with 20 ml Binding buffer (25 mM Hepes, pH7.2, 1 mM CaCl₂) before 50 ml cell culture media with expressed Neprilysin-Fc (IgG2), Neprilysin-Fc(IgG4) or IDE-Fc(IgG4) was applied on the column. Flow rate was approx 1 ml/minute. The columns were washed with 50 ml Binding buffer before the bound protein was eluted with 3.5 M MgCl₂ in water. The elution was time-dependent, and the column was incubated for approx 15 minutes between every column volume of elution buffer. Purified fractions were pooled, and buffer of the pooled protein was exchanged to 50 mM Tris-HCl, pH 7.5, 150 mM NaCl using centrifuge filters (Amicon, Mw cut off 5 kDa). Purified protein was analyzed on SDS-PAGE, and was found to be approximately 80% pure.

### Example 17

### Western Blot Analysis of Expression of Neprilysin, Neprilysin-Fc(IgG4) and Neprilysin-Fc(IgG2)

Cell culture media from expression in mammalian cells was analyzed using western blot. 15 μl cell culture media was diluted in 4×LDS Sample Buffer (Invitrogen) including extra glycerol (5%, final concentration) and DTT (10%, final concentration). The samples were heated to 75° C. for 10 minutes and loaded on an SDS-PAGE gel (4-12% Gradient gel, 10 wells (1 mm), Invitrogen). MES Buffer was used as running buffer. As positive control, commercial Neprilysin (R&D Systems) was used (approx 0.7 μg was loaded on the gel. The gels were run at 200V for 30 minutes.

Electro blotting was performed at 30 V for 1 hour, to transfer the proteins to PVDF membranes. The membranes were blocked in TBST (TBS (20 mM Tris, 500 mM NaCl, pH 7.5 BioRad) plus 0.05% Tween-20). over night before they were incubated with 45 μl primary antibody (Anti-Nep, biotinylated (R&D Systems)) in 15 ml TBST. The membranes were incubated in room temperature for one hour and washed three times with TBST, and incubated for one hour with HRP-conjugated streptavidin (GE Healthcare, diluted 1:10 000 (1.5 μl in 15 ml TBST)). The membranes were wash three times with TBST and three times with water before the bands were visualized using ECL plus reagent (GE Healthcare) and ECL films (GE Healthcare). A typical result is shown in FIG. 19.

### Example 18

### Neprilysin Enzyme Activity Fret-Assay

The Neprilysin enzymatic activity is determined in a fluorescence resonance energy transfer (FRET) assay. Recombinant Human Neprilysin (R&D Systems), culture medium from Neprilysin/Neprilysin-Fc producing cells (AZ Södertälje) or purified Neprilysin/Neprilyusin-Fc was added into 96-well plate containing fluorogenic peptide substrate V-Mca-Arg-Pro-Gly-Phe-Ser-Ala-Phe-Lys(Dnp)-OH(R&D Systems) (SEQ ID NO 52). The final concentration of the control recombinant human Neprilysin was 0.25 μg/ml (and different concentration of the various Neprilysin constructs) and the final concentration of the peptide substrate was 10 μM. 10 μM of Neprilysin inhibitor phosphoramidone (BIOMOL) was added into some wells in order to control the specificity of the signal in the assay and verify the specific Neprilysin activity. Following addition of all components to wells, plate was immediately placed into a fluorescent plate reader (Ascent) and signal was recorded for every minute for 20 minutes at the excitation 340 nm and emission 405 nm. The activity of enzyme was evaluated by calculating the velocity of reaction−Slope coefficient=Σ ΔRFU/Δt. This slope coefficient was used for accurate comparison between the various Neprilysin constructs and productions and compared to a control sample of Neprilysin.

### Example 19

### Degradation of Amyloid β Peptide₁₋₄₀ and Amyloid β Peptide₁₋₄₂ in Guinea Pig Plasma by Neprilysin

Degradation of amyloid β peptide₁₋₄₀ (Aβ₄₀) and amyloid β peptide₁₋₄₂ (Aβ₄₂) by neprilysin was investigated using heparinized plasma from male Dunkin Hartley guinea pigs, weighing 250-300 g (HBLidköping ka). Blood was withdrawn from anaesthetized guinea pigs by heart puncture. The blood were collected into prechilled heparin-plasma tubes and centrifuged for 10 min at 4° C. at 3000×g within 20 minutes of sampling. Plasma samples were transferred to pre-chilled polypropylene tubes and immediately frozen on dry ice and stored at −70° C. prior to use. The experiments were performed on a pool of plasma from 5-6 guinea pigs.

Human recombinant Neprilysin in a buffer containing 25 mM Tris and 100 mM NaCl pH 8 (R&D Systems) or buffer only (i.e. vehicle) was incubated with guinea pig plasma at 37° C. for 0-360 minutes in presence or absents of 10 microM phosphoramidon (Biomol). A final concentration of 4.7 mM EDTA was added into the tubes before the amount of Aβ₁₋₄₀ or Aβ₁₋₄₂ was analyzed by using commercial ELISA kits obtained from Biosource (Aβ₁₋₄₀) or Innogenetics (Aβ₁₋₄₂).

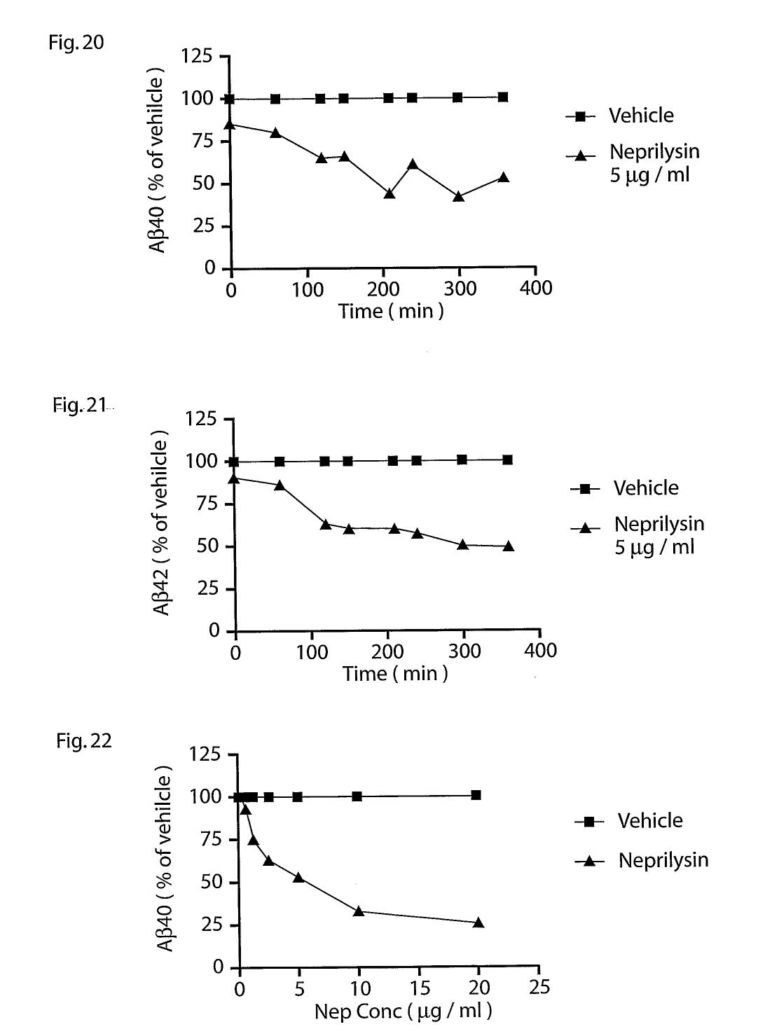

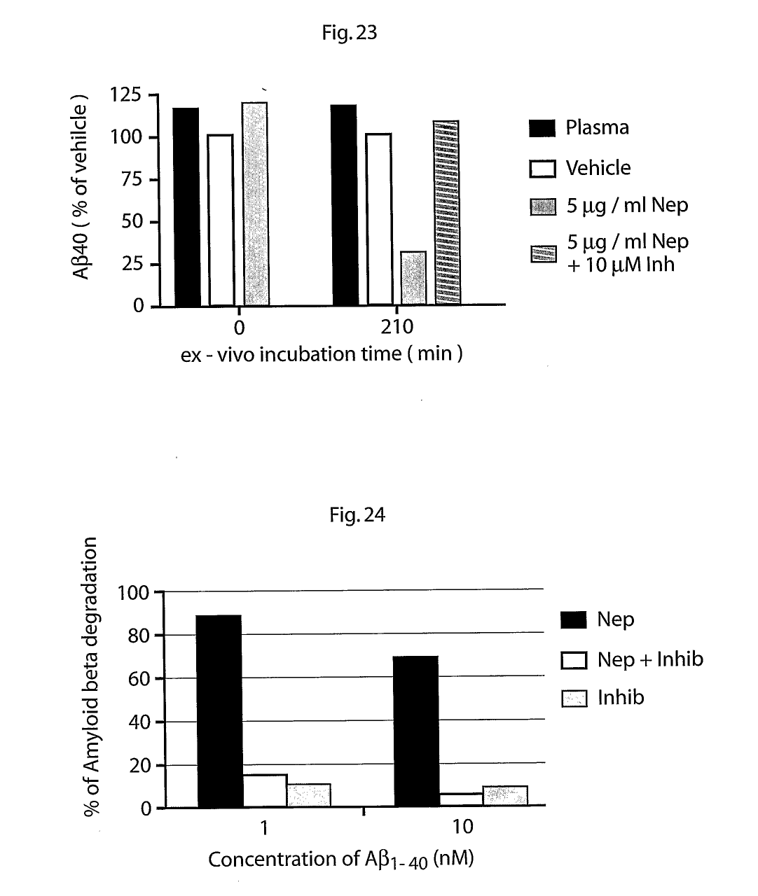

Neprilysin degrades Aβ₁₋₄₀ and Aβ₁₋₄₂ in a time-dependent manner (FIG. 20, FIG. 21). Neprilysin degrades Aβ₁₋₄₀ in a dose dependent manner (FIG. 22). The degradation of Aβ₄₀ is inhibited by addition of 10 microM Phosphoramidon (FIG. 23).

### Example 20

### Degradation of Amyloid β Peptide by Soluble Neprilysin

The goal of this experiment was to demonstrate that Neprilysin is capable to degrade amyloid β1-40 peptide. The assay is measuring the remaining amyloid β1-40 peptide (Bachem) concentration following its incubation in the presence of Neprilysin (R&D Systems) with or without Neprilysin inhibitor. 100 μl of reaction mixture containing of amyloid β1-40 peptide (final concentration 1 or 10 μM) and/or Neprilysin (1.8 μg/ml), and/or Phosphoramidone (10 μM) was incubated in a round bottom 96-well polypropylene plate at 37° C. for 3 hours. Following incubation, 50 μl of antibodies solution containing anti-Aβ40 (final concentration 0.125 μg/ml; Biosource) and Biotinylated 6E10 (final concentration 0.125 μg/ml; Signet) antibodies was added to each well. Plate was incubated at RT for 3 h. Following incubation, 50 μl of detection mixture of Dynabeads M280 (Dynal Biotech ASA) and secondary antibody Ru-GαR (final concentration 0.132 μg/ml) was added to all wells. Plate was incubated on a shaker at RT for 1 h and results were recorded on IGEN/Bio Veris M8 analyser. Amyloid β1-40 peptide degradation by Neprilysin was calculated as a percentage of Amyloid β1-40 peptide left after incubation in the presence of Neprilysin compared to the amyloid β1-40 peptide concentration in the absence of Neprilysin.

Recombinant human Neprilysin at the concentration of 1.8 μg/ml degraded 88% of Amyloid β1-40 peptide (1 nM) after 3 hours incubation at 37° C. This Neprilysin activity was completely abolished in the presence of 10 microM Phosphoramidone (FIG. 24). This example shows that Neprilysin effectively degrade the amyloid β peptide and even a trend towards higher efficiency at a lower Aβ peptide concentration.

### Example 21

### Measurement of Neprilysin Concentration in Cell Culture Supernatant

Neprilysin concentration in cell culture supernatant was measured using Gyros™ Bioaffy™ CD microlaboratory method and Gyrolab Workstation LIF equipment (Gyros AB, Sweden). The goal of the experiment was to identify the optimal conditions for a production of Neprilysin. Samples from different cell cultures were diluted in 1:10 in Sample Diluent (Gyros AB) and placed into Thermo-Fast© 96-well PCR plate (Abgene, UK). Monoclonal mouse biotinylated anti-human Neprilysin antibody (Serotec) was used as a capturing reagent (final concentration 0.05 mg/ml) and polyclonal goat anti-human Neprilysin antibody (R&D Systems) labeled with Alexa Fluor 647 dye (Molecular Probes) served as a detection antibody (final concentration 100 nM). Commercial Neprilysin (R&D Systems) was used as a standard in a concentration range from 31.6 nM to 200 nM in order to construct a standard curve. Standards, capturing and detection antibodies were placed to another Thermo-Fast© 96-well PCR plate (Abgene). Both plates as well as Gyrolab Bioaffy™ 1 CD were placed into Gyrolab Workstation LIF instrument and concentration measurement performed according to the manufacturers protocol using Gyrolab Bioaffy™ Software Package Version 1.8 (Gyros AB). This assay can also be used on purified or partially purified Neprilysin or Neprilysin fusion construct.

### Example 22

### Degradation of Amyloid β Peptide₁₋₄₀ and Amyloid β Peptide₁₋₄₂ in Guinea Pig Plasma By in-House Produced Nep/Fc-Nep (In Vivo Studies)

### Experimental Procedures

In vivo studies in guinea pigs are performed in order to test the in-house produced Nep/Fc-Nep on plasma soluble Aβlevels. The γ-secretase inhibitor, ZR-M550426 (M550426) is used as reference (positive control for reduction of plasma Aβ levels).

The guinea-pigs are weighed and i.v. administrated the appropriate doses. Observations of the animal health are made at the same time points. 6 animals are sacrificed after 0 h, 3 h and 24 h (only Fc-Nep), respectively. The animals are anaesthetized with Isoflurane and blood is sampled by heart puncture. For information about blood sample handling and analysis of soluble Aβ₁₋₄₀ or Aβ₁₋₄₂ (See Example 19). All plasma samples will be sent for PK studies to determined drug exposure (See Example 23).

Study Design for in-House Produced Nep

| | |
|---|---|
| Animal | Male Dunkin Hartley Guinea pigs |
| | 250-300 g |
| Doses | Administer doses of protein that gives |
| | plasma concentrations of 0, 20, 200 μg/ml |
| | after 3 hrs (will be determined by |
| | PK analysis) |
| Route and frequency of | Intra-venous, single dose |
| administration | |
| Time points | 3 hrs |
| Number of animals per group | 6 (3 for blank and 3 for reference, resp.) |
| Number of groups | 5 |
| Total number of animals | 24 |
| Ethical approval No | S58-04 |
| Read out | Soluble Aβ40/42 in plasma by ELISA |
| | (See Example 19) as well as drug |
| | concentration (See Example 23). |

Study Design for in-House Produced Fc-Nep

| | |
|---|---|
| Animal | Male Dunkin Hartley Guinea pigs |
| | 250-300 g |
| Doses | Administer doses of protein that gives |
| | plasma concentrations of 0, 20, 200 μg/ml |
| | after 3 and 24 hrs |
| Route and frequency of | Intra-venous, single dose |
| administration | |
| Time points | 3, 24 hours |
| Number of animals per group | 6 (3 for blank and 3 for reference, resp.) |
| Number of groups | 8 |
| Total number of animals | 42 |
| Ethical approval No | S58-04 |
| Read out | Soluble Aβ40/42 in plasma by ELISA |
| | as well as drug concentration. |

### Example 23

### Pharmacokinetics of Nep-Fc and Neprilysin Only

The Nep-Fc fusion protein was developed to improve the pharmacokinetic entities of neprilysin with the specific aims to reduce clearance and improve half-life. To test, one administers a single i.v. dose in 6 guinea pigs (3 Nep-Fc and 3 neprilysin) as described below. After acclimatization in the animal unit two catheters are inserted in each animal: one in the carotid artery and one in the jugular vein. Following recovery from surgery, typically less than a week, a baseline sample is drawn from the catheter inserted in the jugular vein just prior to the dose. One dose of 1 mg/kg active compound is given i.v. via the catheter in the carotid artery. Blood samples of 150 μl are drawn at 1, 2, 3, 4, 6, 8, 24, 48, 72, 96, 168, 216, 264 and 336 hours after the dose via the catheter inserted in the jugular vein. Upon sampling into tubes containing anticoagulant the aliquots are put on ice. Plasma is prepared by centrifugation within 15 minutes of sampling (typically 1500 g at 4° C. for 10 min) and immediately frozen. Plasma concentrations of Nep-Fc and neprilysin are determined via immunoassays using capture and detection antibodies as described in example 21 or via enzyme-linked immunosorbent assay (ELISA). Pharmacokinetic parameters are calculated using a software package (WinNonlin, Pharsight Corporation, USA).

### Example 24

### IDE-Fc Purification and Enzyme Activity Using the Fret-Assay

IDE-Fc is purified using Protein A sepharose (GE Healthcare). 100 μl protein A sepharose was added to 50 ml cell supernatant and incubated at 4° C. for 6 hours. After incubation, the sepharose was washed once with PBS and resuspended in 1200 μl 50 mM Tris-HCl, pH 7.5, 150 mM NaCl. 600 μl was used for activity measurement and the other 600 μl slurry was pelleted and resuspended in 60 μl 0.1 M citrate, pH 3.2, and incubated for 10 minutes in room temperature. The sepharose was pelleted and the concentration of purified protein in the supernatant was measured by absorbance at 280 nm. The supernatant was analysed by SDS-PAGE and Western blot. 15 μl was diluted in 4×LDS Sample Buffer (Invitrogen) including extra glycerol (5%, final concentration) and DTT (10%, final concentration). The samples were heated to 75° C. for 10 minutes and loaded on an SDS-PAGE gel (4-12% Gradient gel, 12 wells (1 mm), Invitrogen). MES Buffer was used as running buffer. As positive control, commercial IDE (R&D Systems) was used (0.1 μg was loaded on the gel). The gels were run at 200V for 35 minutes. The SDS-PAGE was stained in SyproRuby stain (Molecular Probes) over night and fixed in 50% methanol, 7% acetic acid for 30 minutes.

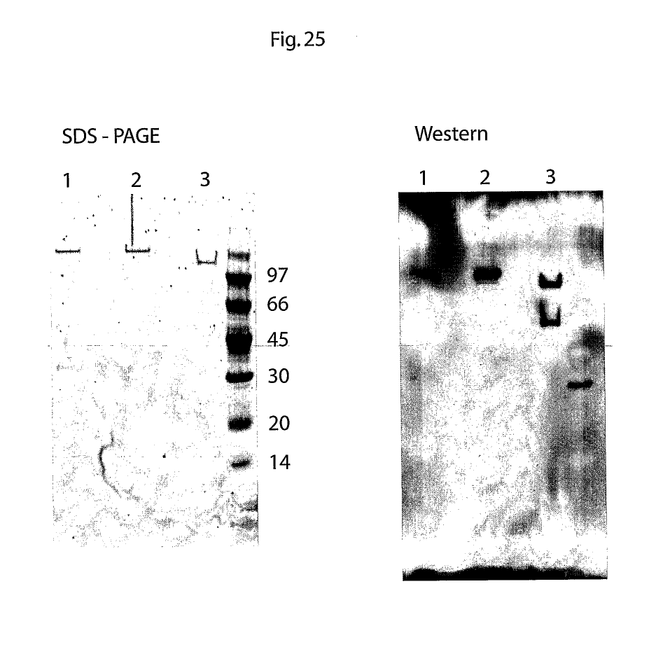

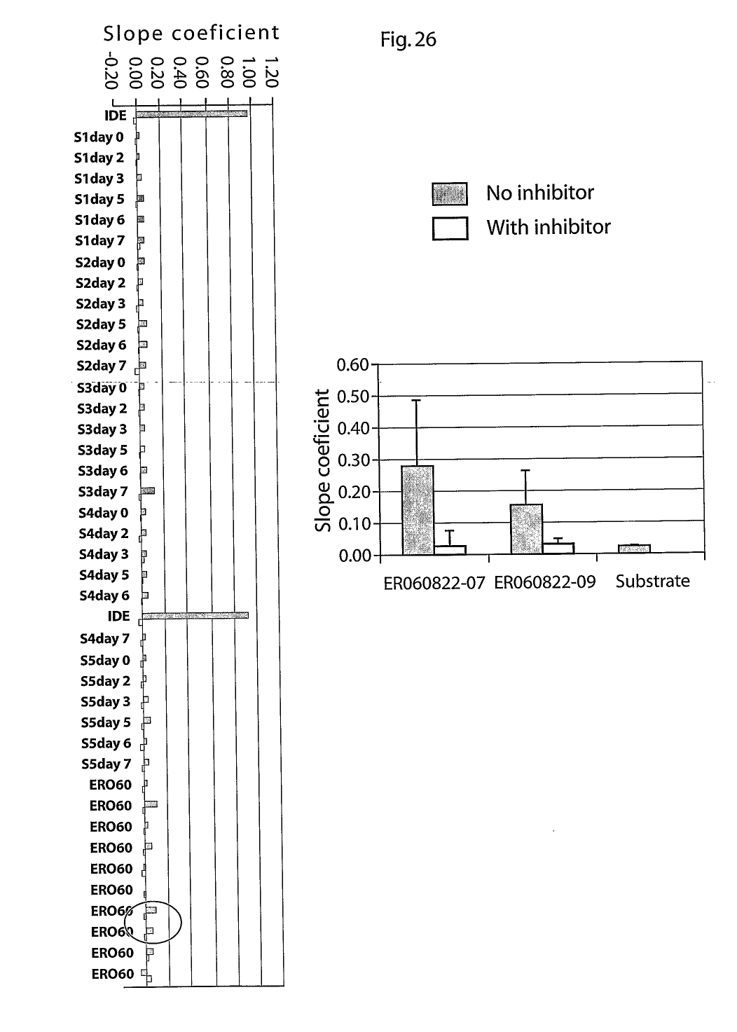

Electro blotting was performed at 15 V for 20 minutes, to transfer the proteins to PVDF membranes. The membranes were blocked in 5% Non-fat dry milk (BioRad) diluted in PBS plus 0.05% Tween-20 (PBST) over night before they were incubated with 0.2 μg/ml primary antibody (Anti-IDE (R&D Systems)) in 10 ml PBST. The membranes were incubated in room temperature for two hours and washed three times with PBST, and incubated for one hour with HRP-conjugated anti-goat antibody (Jackson ImmunoResearch Laboratories). The membranes were wash three times with PBST before bands were visualized using ECL plus reagent (GE Healthcare) and ECL films (GE Healthcare). SDS-PAGE and Western blot on the purified IDE-Fc is shown in FIG. 25. IDE enzymatic activity was to evaluate in a fluorescence resonance energy transfer (FRET) assay. 60 μl of recombinant human IDE (R&D Systems) or sepharose purified culture medium from IDE-Fc producing cells (diluted 1:2 with HEPES buffer) (AZ Södertälje) was added into 96-well plate containing 30 μl of fluorogenic peptide substrate V-Mca-Arg-Pro-Gly-Phe-Ser-Ala-Phe-Lys(Dnp)-OH(R&D Systems) (SEQ ID NO 52). The final concentration of the recombinant human IDE was 0.1 μg/ml and the final concentration of the substrate was 10 μM. 10 μl of (1 mM) of IDE inhibitor phenanthroline (Sigma-Aldrich) was added into some wells in order to control the specificity of the signal. Following addition of all components to wells, plate was immediately placed into a fluorescent plate reader (Ascent) and signal was recorded for every minute for 20 minutes at the excitation 340 nm and emission 405 nm. The activity of enzyme was evaluated by calculating the velocity of reaction−Slope coefficient=Σ ΔRFU/Δt. Enzymatic activity data on purified IDE-Fc are shown in FIG. 26.

### Example 25

### Polymorphism Variants for IDE and ECE

IDE (insulin degrading enzyme) is a 1018 amino acid long protein (SEQ ID No 19). There are splice variants and polymorphism variants described of IDE. In one splice variant, one exon (15a) is replaced with another exon of the same size (15b), encoding a peptide sequence similar to the 15a exon (splice variant (15b) is described in SEQ ID No 20). This variant has been described to be less efficient in degrading both insulin and Aβ. There are also several polymorphisms in the IDE gene described, that lead to amino acid difference identified in this domain: D947N, E612K, L298F and E408G. All combination of these polymorphisms are also possible.

The extra-cellular domain of ECE1 (endothelin-converting enzyme 1) (SEQ ID 26) is a 681 amino acids long protein, defined as amino acid 90-770 of the full-length, membrane-bound ECE1 protein. The ECE1 gene contains several possible polymorphisms that lead to amino acid difference: R665C, W541R, L494Q and T252I. All combinations of these polymorphisms are also possible.

1. A fusion protein having the formula A-L-M capable of degrading amyloid beta peptide at one or more cleavage sites in its amino acid sequence, wherein A is a component that cleaves the amyloid beta peptide; M is a component that modulates the half-life in plasma; and L is a component that connects A and M.

2. The fusion protein according to claim 1, wherein L covalently connects A and M.

3. The fusion protein according to claim 1, wherein A is a protease.

4. The fusion protein according to claim 3, wherein A is improved protease.

5. The fusion protein according to claim 1, wherein A is scaffold protein.

6. The fusion protein according to claim 1, wherein A is human Neprilysin.

7. The fusion protein according to claim 6, wherein said Neprilysin is extracellular Neprilysin.

8. The extracellular Neprilysin according to claim 7, comprising an amino acid sequence according to any one of SEQ ID NO. 1, 2, 3 or 4.

9. The fusion protein according to claim 1, wherein A is insulin degrading enzyme.

10. The fusion protein according to claim 1, wherein M is a Fc part of an antibody.

11. The fusion protein according to claim 10, wherein M is an Fc part from an IgG antibody.

12. The fusion protein according to claim 1, wherein M is selected from pegylation and glycosylation.

13. The fusion protein according to claim 1, wherein L is selected from a peptide, a chemical linker and a direct connection between A and M.

14. The fusion protein according to claim 1, wherein A is human Neprilysin; M is an Fc part from an IgG antibody; and L is a peptide.

15. The fusion protein according to claim 1, comprising the amino acid sequence according to SEQ ID NO. 8.

16. The fusion protein according to claim 1, wherein the combination of component A and component M connected through component L possesses a longer half-life than component A alone.

17. A method for reducing amyloid β peptide concentration, said method comprising administration of a fusion protein, according to claim 1.

18. A method according to claim 17, wherein reducing amyloid β peptide is accomplished in plasma.

19. A method according to claim 17, wherein reducing amyloid β peptide is accomplished in CSF.

20. A method according to claim 17, wherein reducing amyloid β peptide is accomplished in CNS.

21. A pharmaceutical composition capable of degrading amyloid β peptide, comprising a pharmaceutically acceptable amount of fusion protein according to claim 1 together with a pharmaceutically acceptable carrier or excipient.

22. A method of prevention and/or treatment of a condition wherein degradation of amyloid β peptide is beneficial, comprising administering to a mammal, including man in need of such prevention and/or treatment, a therapeutically effective amount of a fusion protein according to claim 1.

23. The method according to claim 22, wherein said condition is Alzheimer's disease or cerebral amyloid angiopathy (CCA).

24-26. (canceled)


SEQUENCE LISTING (52 sequences)

SEQ ID NO: 1 - protein, 699 aa, Homo sapiens
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 2 - protein, 699 aa, Homo sapiens
YDDGICKSSDCIKSAARLIQNMDATTEPCRDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 3 - protein, 699 aa, Homo sapiens
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMRLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 4 - protein, 699 aa, Homo sapiens
YDDGICKSSDCIKSAARLIQNMDATTEPCRDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMRLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 5 - protein, 21 aa, Artificial Sequence
  Signal sequence
METDTLLLWVLLLWVPGSTGD

SEQ ID NO: 6 - protein, 12 aa, Artificial Sequence
  Hinge region from IgG4
ESKYGPPCPSCP

SEQ ID NO: 7 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 949 aa, Artificial Sequence [sequence omitted]

SEQ ID NO: 9 - DNA, 21 bp, Artificial Sequence
  Primer
gtatacgatgatggtatttgc

SEQ ID NO: 10 - DNA, 46 bp, Artificial Sequence
  Primer
ggcatgggggaccatatttggactcccaaacccggcacttcttttc

SEQ ID NO: 11 - DNA, 25 bp, Artificial Sequence
  Primer
ggcatgggggaccatatttggactc

SEQ ID NO: 12 - DNA, 48 bp, Artificial Sequence
  Primer
tccagaaaagaagtgccgggtttgggagtccaaatatggtcccccatg

SEQ ID NO: 13 - DNA, 25 bp, Artificial Sequence
  Primer
tccagaaaagaagtgccgggtttgg

SEQ ID NO: 14 - DNA, 52 bp, Artificial Sequence
  Primer
ggggaccactttgtacaagaaagctgggtctcatttacccagagacagggag

SEQ ID NO: 15 - DNA, 33 bp, Artificial Sequence
  Primer
ggggaccactttgtacaagaaagctgggtctca

SEQ ID NO: 16 - DNA, 77 bp, Artificial Sequence
  Primer
ggggacaagtttgtacaaaaaagcaggcttctttaactttaagaaggagatataaccatggagacagacacactcct

SEQ ID NO: 17 - DNA, 60 bp, Artificial Sequence
  Primer
ggggacaagtttgtacaaaaaagcaggcttctttaactttaagaaggagatataaccatg

SEQ ID NO: 18 - DNA, 21 bp, Artificial Sequence
  Primer
gtcaccagtggaacctggaac

SEQ ID NO: 19 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLMSDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTVMSKLWFKQDDKKKKPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQSLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 20 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLISDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQGWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTAMSKLWFKQDDKFFLPKACLNFEFFSRYIYADPLHCNMTYLFIRLLKDDLKEYTYAARLSGLSYGIASGMNAILLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQGLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 21 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLMSDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTVMSKLWFKQDDKKKKPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQSLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVNAPRRHKVSVHVLAREMDSCPVVGEFPCQNDINLSQAPALPQPEVIQNMTEFKRGLPLFPLVKPHINFMAAKL

SEQ ID NO: 22 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLMSDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTVMSKLWFKQDDKKKKPKACLNFEFFSPFAYVDPLHCNMAYLYLELLKDSLNEYAYAAKLAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQSLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 23 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLMSDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHFKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTVMSKLWFKQDDKKKKPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQSLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 24 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLMSDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQGWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTVMSKLWFKQDDKKKKPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQSLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 25 - protein, 1268 aa, Artificial Sequence [sequence omitted]

SEQ ID NO: 26 - protein, 681 aa, Homo sapiens
QYQTRSPSVCLSEACVSVTSSILSSMDPTVDPCHDFFSYACGGWIKANPVPDGHSRWGTFSNLWEHNQAIIKHLLENSTASVSEAERKAQVYYRACMNETRIEELRAKPLMELIERLGGWNITGPWAKDNFQDTLQVVTAHYRTSPFFSVYVSADSKNSNSNVIQVDQSGLGLPSRDYYLNKTENEKVLTGYLNYMVQLGKLLGGGDEEAIRPQMQQILDFETALANITIPQEKRRDEELIYHKVTAAELQTLAPAINWLPFLNTIFYPVEINESEPIVVYDKEYLEQISTLINTTDRCLLNNYMIWNLVRKTSSFLDQRFQDADEKFMEVMYGTKKTCLPRWKFCVSDTENNLGFALGPMFVKATFAEDSKSIATEIILEIKKAFEESLSTLKWMDEETRKSAKEKADAIYNMIGYPNFIMDPKELDKVFNDYTAVPDLYFENAMRFFNFSWRVTADQLRKAPNRDQWSMTPPMVNAYYSPTKNEIVFPAGILQAPFYTRSSPKALNFGGIGVVVGHELTHAFDDQGREYDKDGNLRPWWKNSSVEAFKRQTECMVEQYSNYSVNGEPVNGRHTLGENIADNGGLKAAYRAYQNWVKKNGAEHSLPTLGLTNNQLFFLGFAQVWCSVRTPESSHEGLITDPHSPSRFRVIGSLSNSKEFSEHFRCPPGSPMNPPHKCEVW

SEQ ID NO: 27 - protein, 931 aa, Artificial Sequence [sequence omitted]

SEQ ID NO: 28 - protein, 12 aa, Artificial Sequence
  Hinge region of IgG2
ERKCCVECPPCP

SEQ ID NO: 29 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 948 aa, Artificial Sequence
  Synthetically generated peptide
METDTLLLWVLLLWVPGSTGDYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVWERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - DNA, 23 bp, Artificial Sequence
  Primer
atgcggtaccggctagcgtggct

SEQ ID NO: 32 - DNA, 25 bp, Artificial Sequence
  Primer
tcagagttttgcagccatgaagtta

SEQ ID NO: 33 - DNA, 86 bp, Artificial Sequence
  Primer
atggagacagacacactcctgctatgggtactgctgctctgggttccaggttccactggtgaccggtaccggctagcgtggcttct

SEQ ID NO: 34 - DNA, 42 bp, Artificial Sequence
  Primer
aataccggttctagactcgagttttgcagccatgaagttaat

SEQ ID NO: 35 - DNA, 29 bp, Artificial Sequence
  Primer
attctcgagtccaaatatggtcccccatg

SEQ ID NO: 36 - DNA, 33 bp, Artificial Sequence
  Primer
aataccggttcatttacccagagacagggagag

SEQ ID NO: 37 - DNA, 60 bp, Artificial Sequence
  Primer
ggggacaagtttgtacaaaaaagcaggcttctataaccatggagacagacacactcctgc

SEQ ID NO: 38 - DNA, 54 bp, Artificial Sequence
  Primer
ggggaccactttgtacaagaaagctgggtctcatttacccagagacagggagag

SEQ ID NO: 39 - DNA, 84 bp, Artificial Sequence
  Primer
atggagacagacacactcctgctatgggtactgctgctctgggttccaggttccactggtgaccagtaccagacaagatccccc

SEQ ID NO: 40 - DNA, 39 bp, Artificial Sequence
  Primer
aataccggttctagagaattcgcacttgtgaggcgggtt

SEQ ID NO: 41 - DNA, 34 bp, Artificial Sequence
  Primer
gcgaattctgggagtccaaatatggtcccccatg

SEQ ID NO: 42 - DNA, 34 bp, Artificial Sequence
  Primer
cctcacaagtgcgaagtctgggagtccaaatatg

SEQ ID NO: 43 - DNA, 34 bp, Artificial Sequence
  Primer
catatttggactcccagacttcgcacttgtgagg

SEQ ID NO: 44 - DNA, 31 bp, Artificial Sequence
  Primer
ggggacaagtttgtacaaaaaagcaggcttc

SEQ ID NO: 45 - DNA, 46 bp, Artificial Sequence
  Primer
ggcactcgacacaacatttgcgctcccaaacccggcacttcttttc

SEQ ID NO: 46 - DNA, 45 bp, Artificial Sequence
  Primer
tccagaaaagaagtgccgggtttgggagcgcaaatgttgtgtcga

SEQ ID NO: 47 - DNA, 61 bp, Artificial Sequence
  Primer
ggggaccactttgtacaagaaagctgggtctcatttacccggagacagggagaggctcttc

SEQ ID NO: 48 - DNA, 31 bp, Artificial Sequence; identical to SEQ ID NO: 44
  Primer

SEQ ID NO: 49 - DNA, 54 bp, Artificial Sequence
  Primer
ggggaccactttgtacaagaaagctgggtctcaccaaacccggcacttcttttc

SEQ ID NO: 50 - protein, 43 aa, Artificial Sequence
  Synthetically generated peptide
DAEFRHDSGYEVHHQKLVFFAEDVGSNKGAIIGLMVGGVVIAT

SEQ ID NO: 51 - protein, 43 aa, Artificial Sequence
  Naturally occurring peptide
DAEFRHDSGYEVHHQKLVFFAEDVGSNKGAIIGLMVGGVVIIA

SEQ ID NO: 52 - protein, 8 aa, Artificial Sequence
  Fluorogenic peptide substrate
RPGFSAFK